(19) United States
(12) Patent Application Publication
(10) Pub. No.: US 2025/0123282 A1
(43) Pub. Date: Apr. 17, 2025

(54) METHODS FOR EVALUATION AND TREATMENT OF TYROSINE KINASE INHIBITOR (TKI)-RESISTANT ACUTE MYELOID LEUKEMIA

(71) Applicant: LOMA LINDA UNIVERSITY, Loma Linda, CA (US)

(72) Inventors: Yi (David) Xu, Loma Linda, CA (US); Huynh CAO, Loma Linda, CA (US)

(21) Appl. No.: 18/723,125

(22) PCT Filed: Dec. 22, 2022
(86) PCT No.: PCT/US22/82308

§ 371 Date: Jun. 21, 2024

Related U.S. Application Data
(60) Provisional application No. 63/265,889, filed on Dec. 22, 2021.

(51) Int. Cl. G01N 33/574; A61K 31/17; A61K 31/277; A61K 31/497; A61K 31/522; A61K 31/5377; A61K 31/635; A61K 38/06; A61K 38/08; A61P 35/02

(52) U.S. Cl. CPC G01N 33/57426; A61K 31/17; A61K 31/277; A61K 31/497; A61K 31/522; A61K 31/5377; A61K 31/635; A61K 38/06; A61K 38/08; A61P 35/02; G01N 2333/70503; G01N 2333/70585

(57) ABSTRACT

Disclosed here are methods of evaluating resistance to a tyrosine kinase inhibitor (TKI) therapy of acute myeloid leukemia (AML) in a subject that include detecting the status of CD33, CD44, and phosphorylated BCL2 associated agonist of cell death (pBAD) expression. Also disclosed herein are methods of treating AML in a subject by administering a TKI and one or more inhibitors targeting a TKI-activated compensation pathway to the subject.

### CROSS-REFERENCE TO RELATED APPLICATIONS

This application claims the benefit of and priority to U.S. Provisional Application No. 63/265,889, filed on Dec. 22, 2021, the content of which is incorporated herein by reference in its entirety.

### TECHNICAL FIELD

The disclosure relates to compositions and methods of treatment of cancer utilizing combination of a therapeutic agent and an inhibitor targeting treatment-activated compensation systems.

### BACKGROUND

Acute myeloid leukemia (AML) is a heterogeneous bone marrow malignancy with some of the lowest survival rates among the leukemias. Disease relapse is the most important cause of treatment failure. Standard treatment for AML has been chemotherapy with a combination of anthracyclines and cytarabine. In addition, target therapies for AML are being developed using next generation sequencing and based on identification of genetic mutations. FMS-like receptor tyrosine kinase 3 (FLT3) is expressed in many AMLs, with 35% of AML patients carrying an oncogenic FLT3 mutation, such as the internal tandem duplication (ITD) or a point mutation in the TK domain (TKD) of this protein. FLT3 mutations have been associated with poorer overall survival with an increased risk of relapse in AML.

Mechanistically, constitutive activation of mutated FLT3 results in the stimulation of multiple downstream signaling pathways including PI3K-AKT, RAS-RAF-ERK, and JAK/STAT5 to promote cell survival and proliferation.

Multiple tyrosine kinase inhibitors (TKI) including Midostaurin (MIDO), Sorafenib (SORA), Gilteritinib (GILT), and Quizartinib (QUIZ) have been evaluated in clinical trials for the treatment of AML with FLT3 mutations, some of which have received U.S. Food and Drug Administration (FDA) approval. However, the anti-leukemic efficacy of the latest approved FLT3 inhibitors as monotherapy or in combination with standard treatments are constrained by a short duration of response and a high rate of relapse. Relapsed or refractory AML patients with mutations in FLT3 have poor response to salvage chemotherapy. Even with the new generation of FLT3 inhibitors, the median overall survival is less than one year. Although epigenetic and proteomic profiles of AML have been reported, the specific immunophenotypic biomarkers for TKI-resistant blasts remain to be identified.

### SUMMARY

Provided here are systems and methods to address these shortcomings of the art, including addressing AML relapse after TKI treatment and developing effective therapies to overcome TKI-resistance, and provide other additional or alternative advantages. Disclosed herein are methods of evaluating resistance to a TKI therapy of AML in a subject. One such method includes isolating AML tumor cells from the subject and detecting status of markers CD33, CD44, and phosphorylated BCL2 associated agonist of cell death (pBAD) on surface of the AML tumor cells, an in response to the tumor cells being CD33 positive (CD33⁺), CD44 high positive (CD44high⁺), and pBAD positive (pBAD⁺), the subject is determined to be at an increased risk of developing TKI-resistant AML. In some embodiments, the CD33⁺ CD44high⁺pBAD⁺ tumor cells are also nuclear protein Ki67 positive (Ki67⁺). Also disclosed herein are embodiments of methods of treating acute myeloid leukemia (AML) in a subject. One such method includes administering to the subject a therapeutically effective amount of a TKI and a therapeutically effective amount of an inhibitor of a TKI-activated compensation pathway or an inhibitor of the intracellular homeostasis required for leukemic relapse after treatment. In some embodiments, the inhibitor inhibits a JAK-STAT pathway, a TYK2-STAT4-PIM2/PIM3 pathway, and/or a NFκB2(P100/P52)-MIF-CXCR2 pathway. In some embodiments, the inhibitor is one or more of a STAT4 inhibitor, a BCL2 inhibitor, a CD44 inhibitor, a CXCR2 inhibitor, and an NFκB inhibitor. In some embodiments, the methods include administering a therapeutically effective amount of a TKI, a therapeutically effective amount of a BCL2 inhibitor, and a therapeutically effective amount of a NFκB inhibitor to the subject. In some embodiments, the methods include administering a therapeutically effective amount of a TKI, a therapeutically effective amount of a STAT4 inhibitor, a therapeutically effective amount of a BCL2 inhibitor, and a therapeutically effective amount of a NFκB inhibitor to the subject. In some embodiments, the TKI is GILT or QUIZ. In some embodiments, the STAT4 inhibitor is (R)-Lisofylline. In some embodiments, the BCL2 inhibitor is Venetoclax. In some embodiments, the NFκB inhibitor is MG-132 or BAY 11-7082. In some embodiments, the CD44 inhibitor is a urokinase-derived peptide, Angstrom6. In some embodiments, the CXCR2 inhibitor is SB225002. In some embodiments, the subject has TKI-resistant AML or is at risk of developing TKI-resistant AML.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIGS. 1A-1E depicts therapeutic effect of different TKIs on MV4-11 and MOLM-14 cells in vitro (N=6). FIG. 1A shows representative FACS (fluorescence-activated single cell sorting) plots of different TKI inhibitors and 5-Azacitidine (AZA) and 1α,25-Dihydroxyvitamin D3 (VD3) on MV4-11 cells after 3 days of in vitro treatment. FIG. 1B shows representative FACS plots of different TKI inhibitors and AZA/VD3 on MOLM-14 cells after 3 days of in vitro treatment. The circles indicate the group of viable blasts located in a FACS plot and green arrows indicate the dying tendency of blasts which will become positive for viability dye during the next few days. FIGS. 1C and 1D are graphical representations of the cumulative FACS percentage data of viable MV4-11 and MOLM-14 cells. *P<0.05. FIG. 1E is a representative FACS plot of Ki67 expression in different treatment groups after 3 days of in vitro treatment. Black arrows indicate the expression curve of Ki67, showing treated groups with less expression of Ki67 when compared to the non-treatment control.

FIGS. 2A-2F depict TKI-treated blasts undergoing tumorigenic clustering re-growth after 10 days of in vitro treatment with different TKIs (N=6). FIG. 2A shows representative phase-bright images showing different re-growth of TKI-treated MV4-11 and MOLM-14 cells. The arrows in the images of cells treated with GILT indicate many re-grown tumorigenic clusters in GILT-treated blasts. The arrows in the images of cells treated with QUIZ indicate few re-grown tumorigenic clusters in QUIZ-treated blasts; Scale bar: 100 μm. There are similar patterns of blasts re-growth including timing and immunophenotypes in TKI-treated MV4-11 and TKI-treated MOLM-14 in vitro studies. FIG. 2B shows representative FACS plots of pBAD expression in MIDO and SORA experimental groups after 3 days of in vitro treatment. The expression curve of pBAD in different experimental groups show treated groups with more expression of pBAD in both MV4-11 and MOLM-14 cells when compared to the non-treatment control. FIG. 2C is a set of representative FACS plots of CD44⁺pBAD⁺ populations in different TKI Inhibitors-treated MV4-11 cells after 10 days of their continuous culture in vitro. Arrows indicate CD44⁺pBAD⁺ cells in GILT-treated blasts and QUIZ-treated blasts. The boxes indicate higher expression of CD44 in GILT-treated and QUIZ-treated blasts (“CD44high⁺”). FIG. 2D is a graphical representation of the cumulative FACS percentage data of viable CD44⁺pBAD⁺ MV4-11 cells. *P<0.05. FIG. 2E is a set of representative FACS plots of Ki67⁺CD33⁺CD44⁺ populations in different TKI-treated MV4-11 cells after their continuous culture in vitro. Arrows indicate Ki67⁺CD33⁺CD44⁺ cells in GILT-treated blasts and in QUIZ-treated blasts. Circles indicate groups of Ki67⁺CD33⁺CD44⁺ cells in experimental groups of control, and MIDO- and SORA-treated MV4-11 cells. FIG. 2F is a graphical representation of the FACS percentage data of viable Ki67⁺CD33⁺CD44⁺ MV4-11 cells. *P<0.05.

FIGS. 3A-3E depict GILT-treated MV4-11 cells recovering and growing confluent after 20 days of in vitro treatment (N=6). FIG. 3A is a set of representative phase-bright images showing control and of TKI-treated MV4-11 cells. Arrows indicate tumorigenic clusters in QUIZ-treated blasts. Scale bar: 50 μm. FIG. 3B is a set of representative FACS plots of CD44⁺pBAD⁺ populations in different TKI-treated MV4-11 cells after 20 days of their continuous culture in vitro. Arrows indicates CD44⁺pBAD⁺ cells in GILT-treated blasts and QUIZ-treated blasts. FIG. 3C is a graphical representation of cumulative FACS percentage data of viable CD44⁺pBAD⁺ MV4-11 cells. Arrows indicates GILT-treated blasts and QUIZ-treated blasts. **P<0.01. FIG. 3D is a set of representative FACS plots of Ki67⁺CD33⁺CD44⁺ populations in different TKI-treated MV4-11 cells after 20 days of their continuous culture in vitro. Arrows indicate Ki67⁺CD33⁺CD44⁺ cells in GILT-treated blasts and in QUIZ-treated blasts. FIG. 3E is a graphical representation of cumulative FACS percentage data of viable Ki67⁺CD33⁺CD44⁺ MV4-11 cells; Arrows indicate GILT-treated blasts and QUIZ-treated blasts. **P<0.01.

FIGS. 4A-4M depict qPCR analyses of prosurvival gene expression changes in TKI-treated MV4-11 in vitro (3, 10, and 20 days). After 3, 10, or 20 days of treatment of different TKIs in vitro, the cells were collected for RNA isolation as described in Materials and Methods. The gene expressions were analyzed by qPCR. FIG. 4A is a set of graphical representations of mRNA expressions of the genes necessary for the pathway of cell death at 3 days after treatment. FIGS. 4B and 4C are a set of graphical representations of mRNA expressions of the genes necessary for the phosphorylation of BAD protein 3 days after treatment. FIG. 4D is a set of graphical representations of mRNA expressions of the genes necessary for the signaling pathway of JAK-STAT at 3 days after treatment. FIG. 4E is a set of graphical representations of mRNA expressions of the genes necessary for the intracellular inhibitory pathway at 3 days after treatment. In FIGS. 4A-4E, where applicable, data are means±SEM. *P<0.05, **P<0.01, N=4-6. FIGS. 4F-4I are a set of graphical representations of mRNA expression of genes necessary for the compensation pathways (FIG. 4F and FIG. 4H) and genes necessary for the initialization of intracellular inhibitory pathways (FIG. 4G and FIG. 4I) at 10 days after the treatment of different TKI drugs in vitro. FIGS. 4J-4M are a set of graphical representations of mRNA expression of genes necessary for the compensation pathways (FIG. 4J and FIG. 4L) and genes necessary for the initialization of intracellular inhibitory pathways (FIG. 4K and FIG. 4M) at 20 days after the treatment of different TKI drugs in vitro. In FIGS. 4F-4M, where applicable, data are means±SEM. *P<0.05, N=4.

FIGS. 5A-5D depict QUIZ-treated MV4-11 recovered and grew confluent after 28 days in vitro (N=6). FIG. 5A is a set of representative FACS plots of CD44⁺pBAD⁺ populations in different TKI-treated MV4-11 cells after their continuous culture in vitro. Arrow indicates CD44⁺pBAD⁺ cells in QUIZ-treated blasts. FIG. 5B is a set of representative FACS plots of Ki67⁺CD33⁺CD44⁺ populations in different TKI inhibitors-treated MV4-11 cells after 28 days of their continuous culture in vitro. Arrow indicates Ki67⁺CD33⁺CD44⁺ cells in QUIZ-treated blasts. FIGS. 5C and 5D are graphical representations of cumulative FACS percentage data of viable CD44⁺pBAD⁺ MV4-11 and Ki67⁺CD33⁺CD44⁺ MV4-11 cells; *P<0.05.

FIGS. 6A-6D depict qPCR analyses of pro-survival gene expression changes in TKI-treated MV4-11 in vitro (28 days). Twenty-eight (28) days after the treatment of different TKI drugs in vitro, the cells were collected for RNA isolation as described in Materials and Methods. The gene expressions were analyzed by qPCR. FIG. 6A is a set of graphical representations of mRNA expressions of the genes necessary for the pathway of cell death. FIG. 6B is a set of graphical representations of mRNA expressions of the genes necessary for the phosphorylation of BAD protein. FIG. 6C is a set of graphical representations of mRNA expressions of the genes necessary for the signaling pathway of JAK-STAT. FIG. 6D is a set of graphical representations of mRNA expressions of the genes necessary for the intracellular inhibitory pathway. Where applicable, data are means±SEM. *P<0.05, N=4-6.

FIGS. 7A-7E depict release of macrophage migration inhibitory factor (MIF) from AML blasts. FIG. 7A is a set of images of partial blot films developed for proteomic analyses of cell-free supernatants from 80 nM GILT-treated MV4-11 cells. The dotted arrows indicate Osteopontin at the same location in the film. The hatched arrows indicate the dots of MIF at the same location in the film. Each antibody has two dot spots according to manufacturer's specification. FIG. 7B is a graphical representation of Cumulative Mean Pixel Densities of MIF (Fold Change). **P<0.01. FIG. 7C is a set of images of partial blot films developed for proteome analyses of cell-free supernatants from 80 nM GILT-treated primary AML BMMNC cells (Patient #1). The solid arrows indicate the reference spots (control dots) from the manufacturer. The hatched arrows indicate the dots of MIF at the same location in the film. FIG. 7D is a graphical representation of cumulative mean pixel densities of MIF (fold change). **P<0.01. FIG. 7E is a set of graphical representations of a qPCR gene expression analysis at 3 days after the treatment of different TKI in vitro. Data show mRNA expressions of the genes encoding different receptors CD74, CD44, CXCR4 and CXCR2 for MIF. *P<0.05, **P<0.01. (MIF) in MV4-11.

FIGS. 8A-8G depict MIF promotes the proliferation of MV4-11 through up-regulating the expressions of CXCR2, cytokines and cell division proteins. FIG. 8A is a representative FACS histogram plot of Ki67 expression in MV4-11 experimental groups after 2 days in vitro. Treated groups showed more expression of Ki67 when compared to the non-treatment control. FIG. 8B is representative FACS histogram plot of Ki67 expression in RAW264.7 experimental groups after 2 days in vitro. Treated groups showed less expression of Ki67 when compared to the non-treatment control.

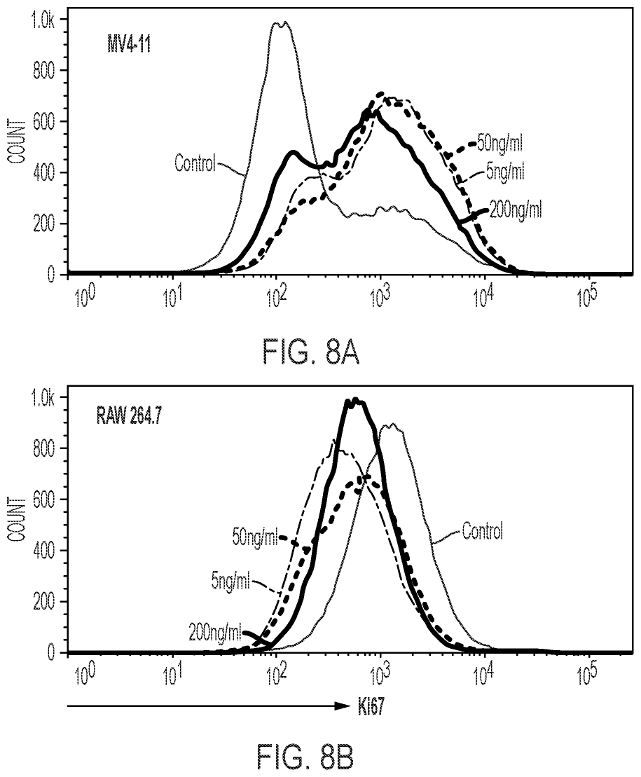

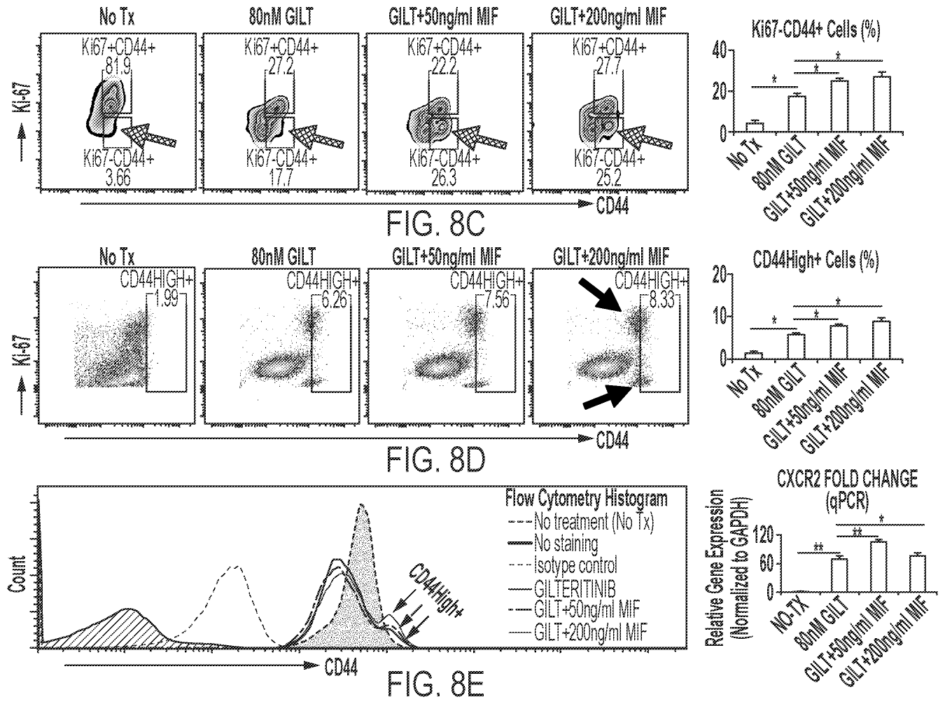

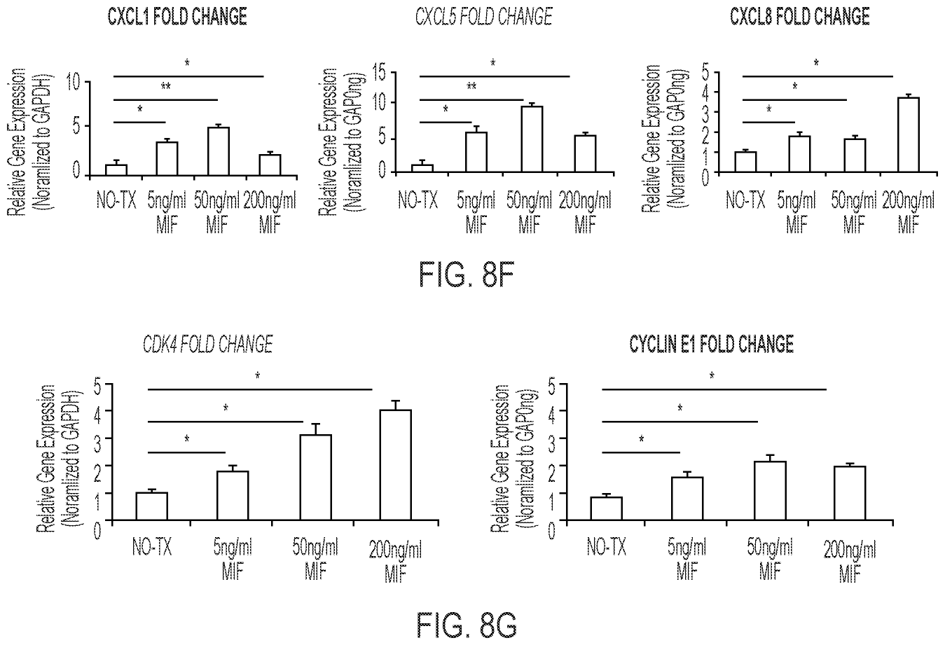

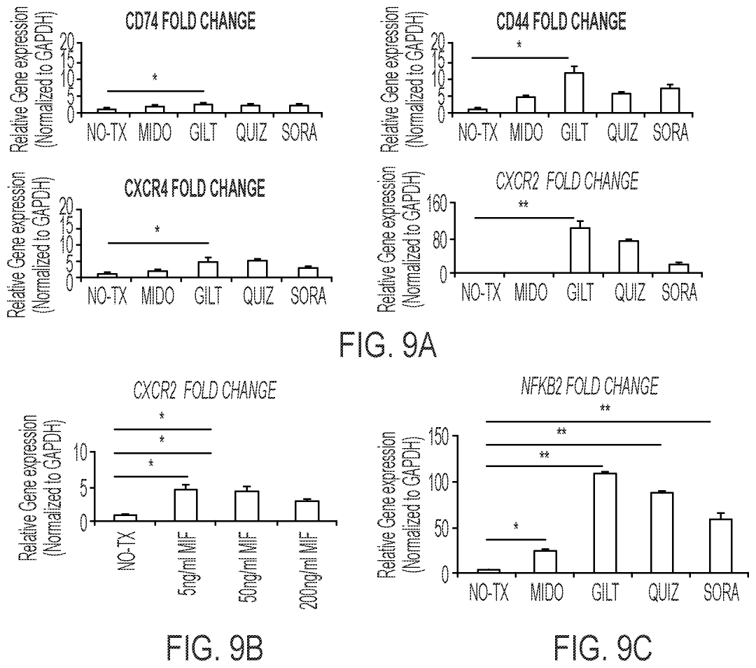

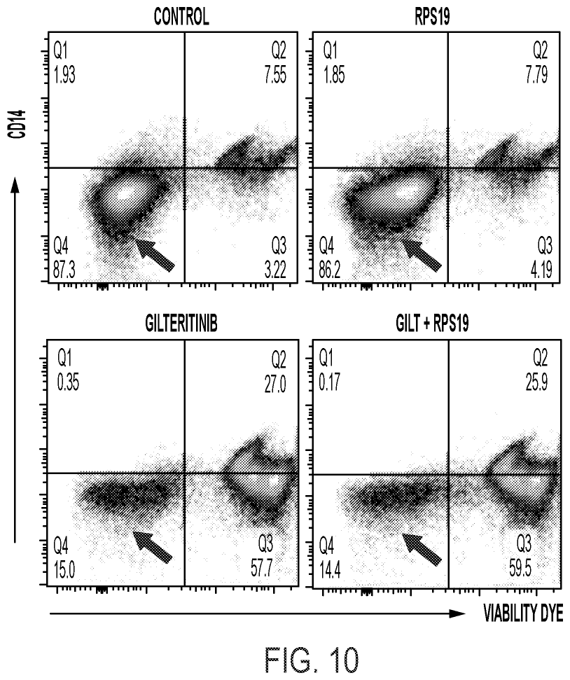

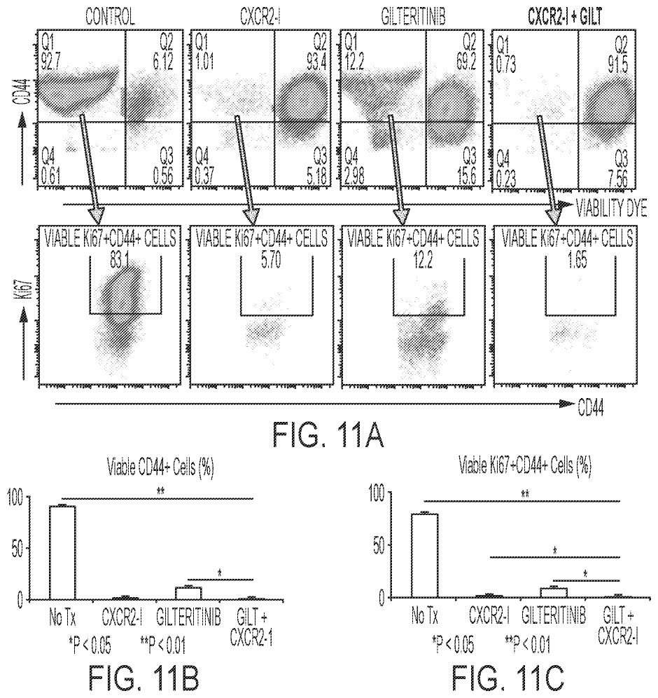

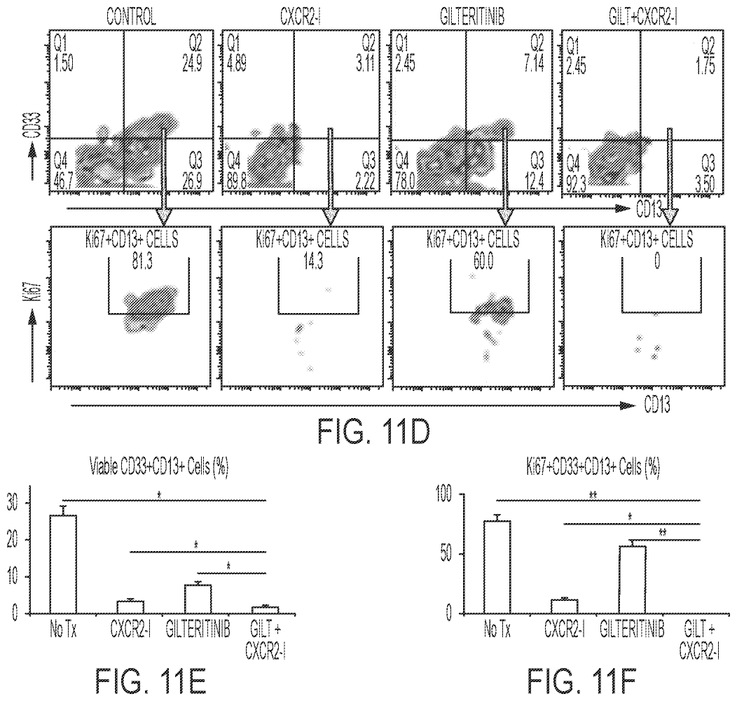

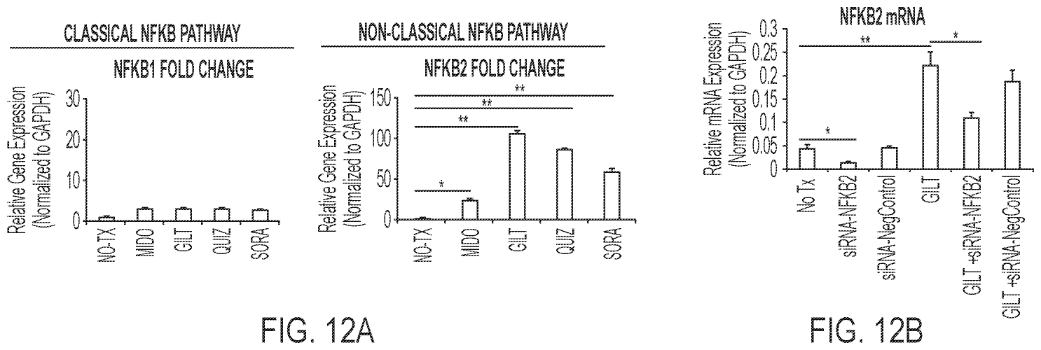

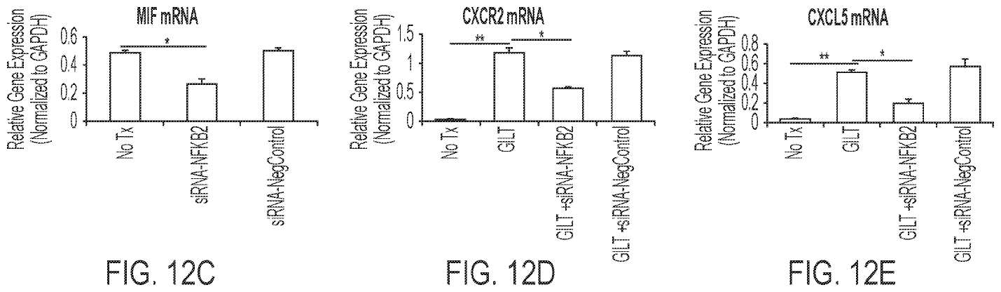

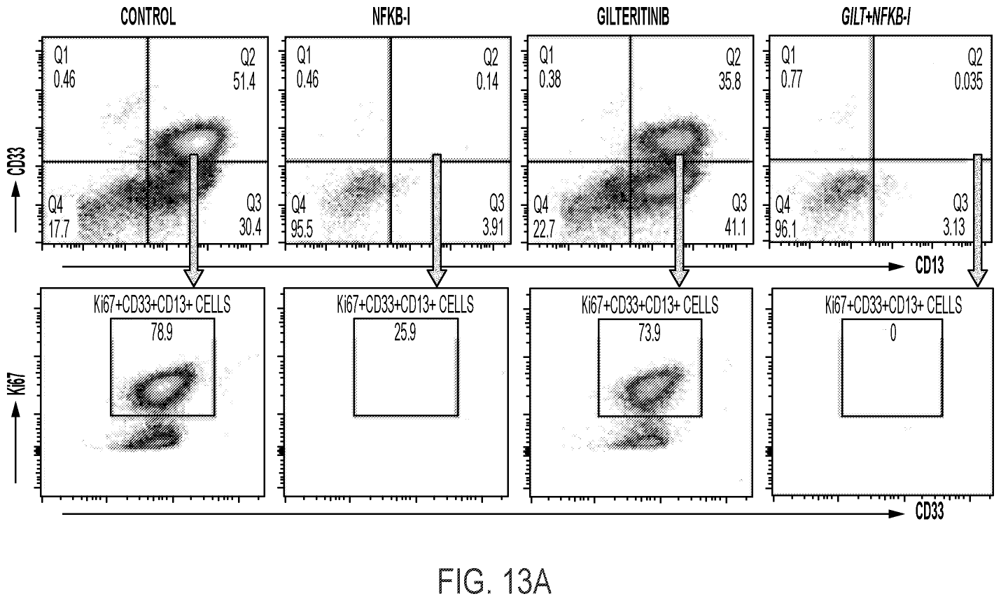

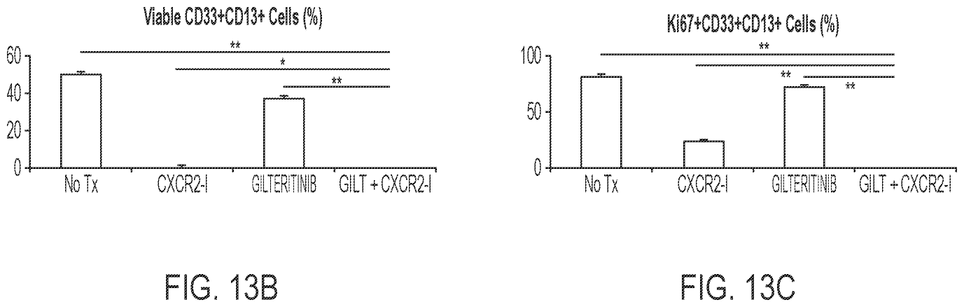

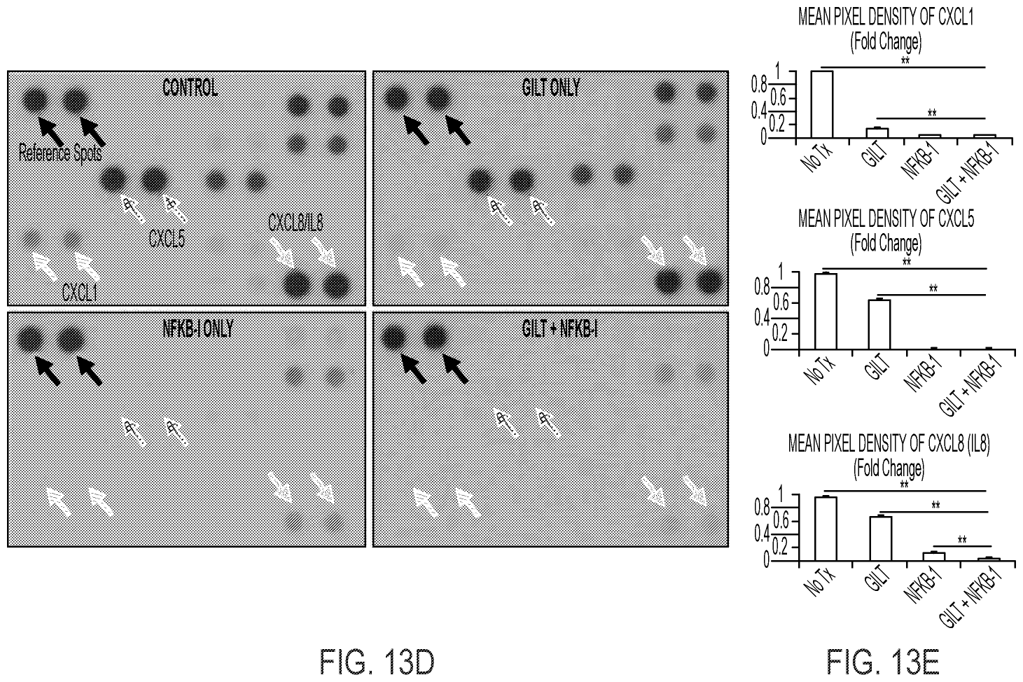

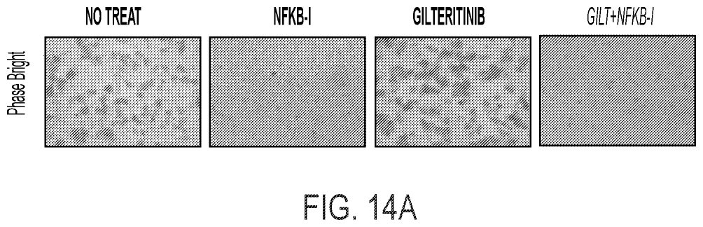

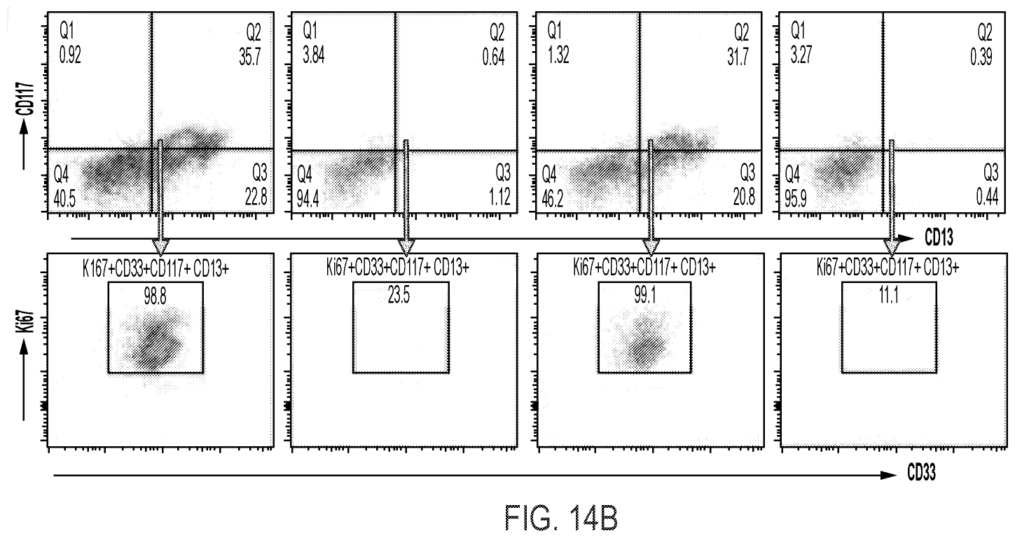

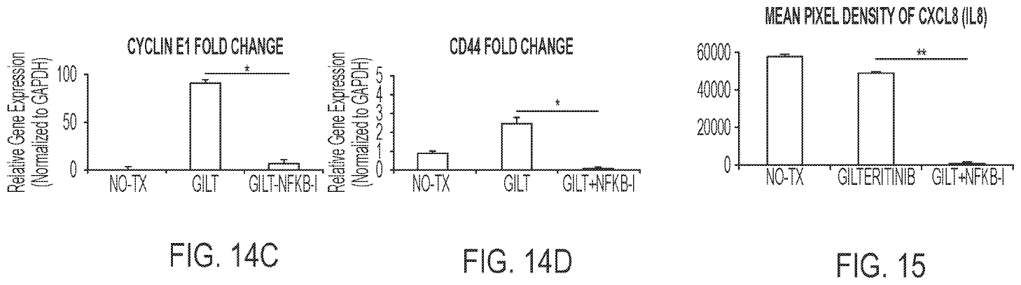

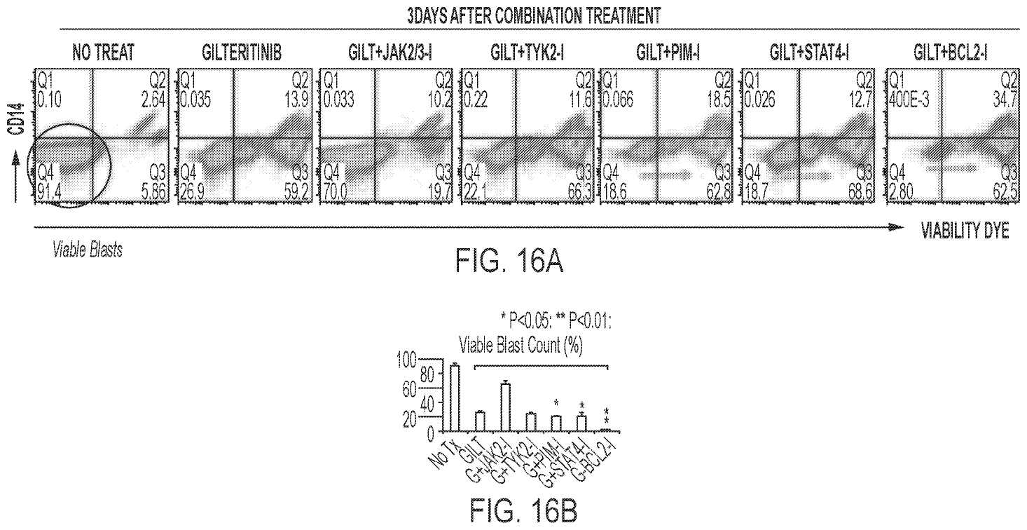

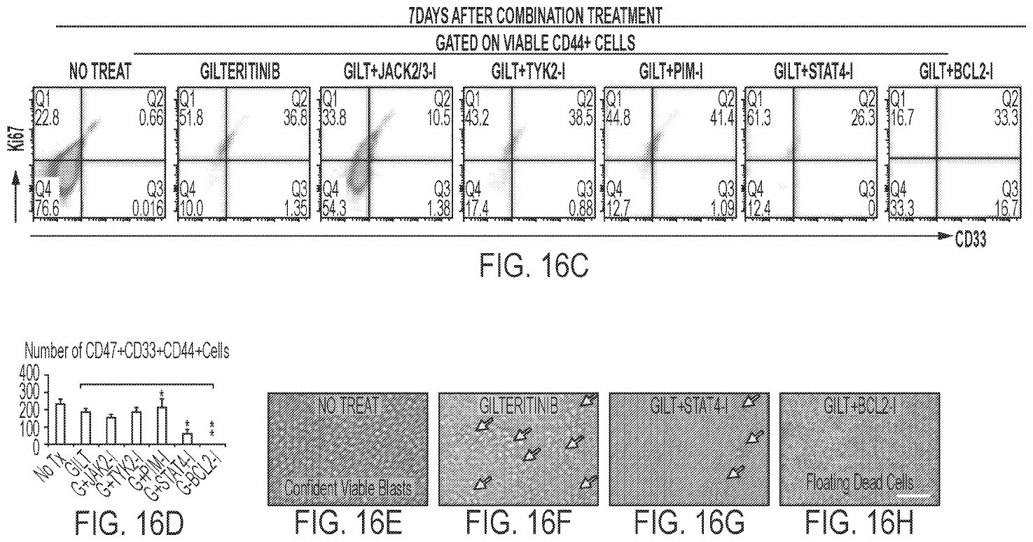

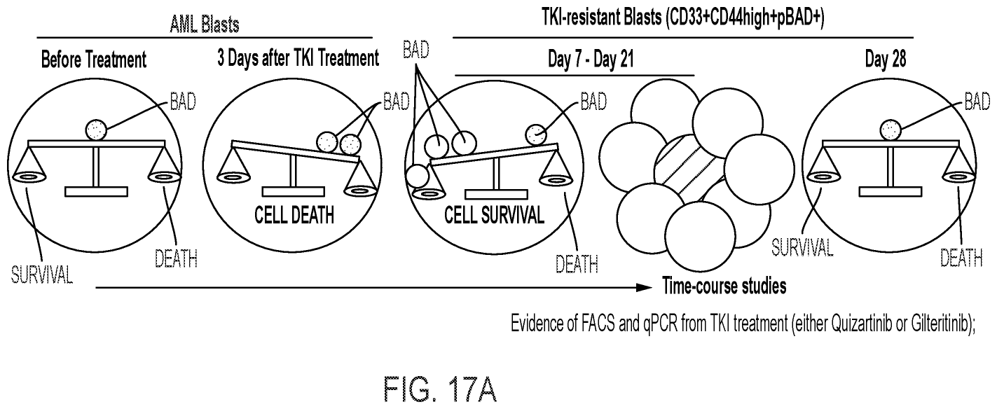

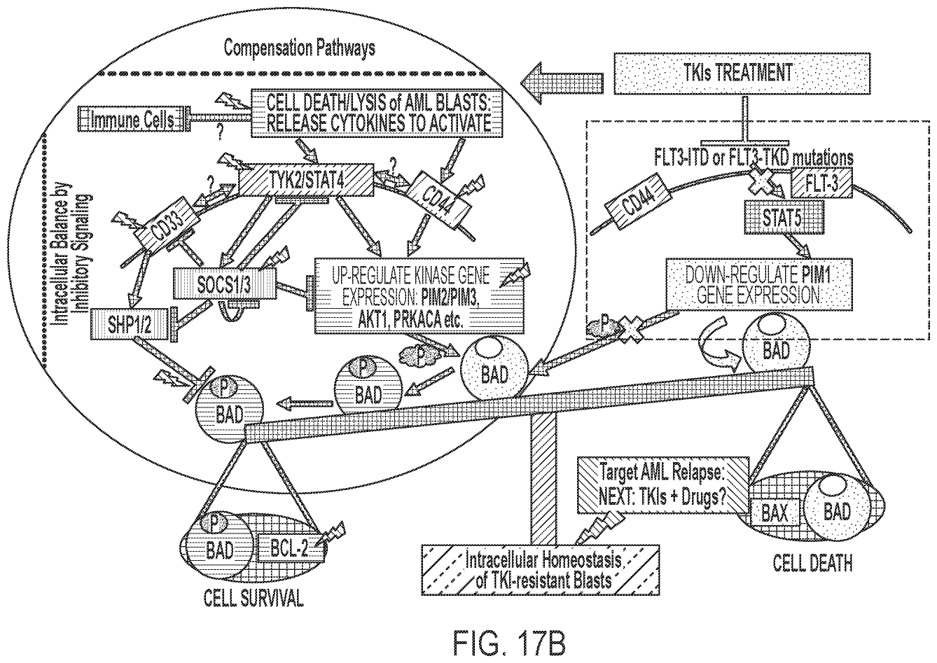

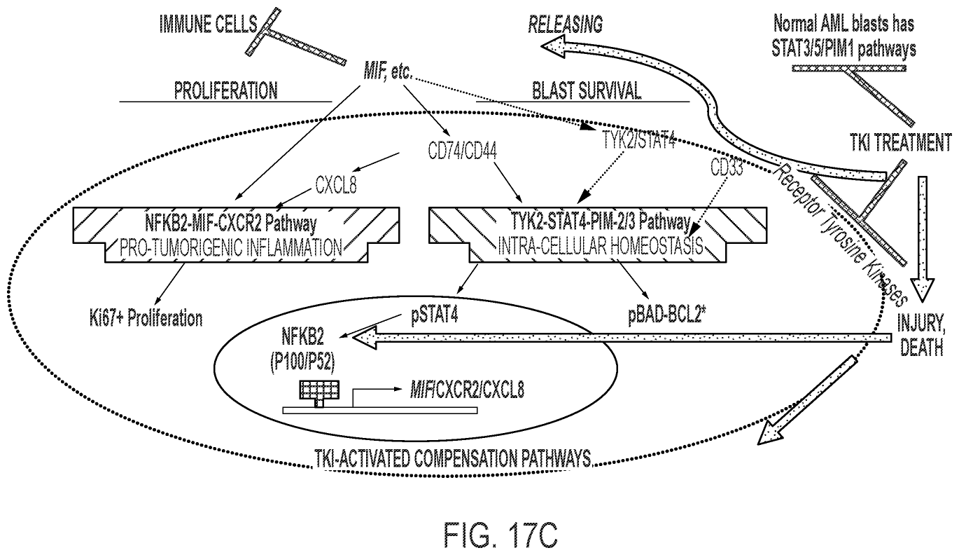

MIF promoted the survival of a group of CD44High+ cells after TKI treatment in vitro. FIG. 8C is a set of representative FC plots of Ki-67 and CD44 expressions in MV4-11 experimental groups after 5 days' sequential coculture of 80 nM GILT and appropriate doses of MIF in vitro; Arrows indicate viable Ki-67-CD44+ cells. FIG. 8C also includes, on the right side, a graphical representation of the Cumulative FC percentage data of viable Ki-67-CD44+ cells. *p<0.05. FIG. 8D is a set of representative FC plots of Ki-67 and CD44 expressions in MV4-11 experimental groups after 5 days' simultaneous coculture of 80 nM GILT and appropriate doses of MIF in vitro; Arrows indicate Ki-67+CD44+ or Ki-67-CD44+ cell population respectively. FIG. 8D also includes, on the right side, a graphical representation of the Cumulative FC percentage data of viable CD44High+ cells. *p<0.05. FIG. 8E is a representative FC histogram plot of CD44 expression in MV4-11 experimental groups after 5 days' simultaneous coculture in vitro; The groups were treated with 80 nM GILT alone or its combination with different doses of MIF. The GILT-treated groups with the supplementation of MIF had a group of CD44High+ cells when compared to the non-treatment control or GILT alone. FIG. 8E also includes, on the right side, a graphical representation of the change of mRNA expression of CXCR2 gene in MV4-11 cells at different doses of MIF combined with 80 nM GILT in vitro. *p<0.05, **p<0.01. FIG. 8F is a set of graphical representations of the change of mRNA expressions of CXCL1, 5, 8 chemokine genes in MV4-11 cells at different doses of MIF in vitro; *P<0.05, **P<0.01. FIG. 8G is a set of graphical representations of the change of mRNA expressions of CDK4 and CYCLIN E1 genes in MV4-11 cells at different doses of MIF in vitro; *P<0.05.

FIGS. 9A-9B depict MIF and TKI treatment-induced CXCR2 expression in MV4-11 cells in vitro. FIG. 9C depicts TKI treatment-induced NFκB2 activation. *P<0.05, **P<0.01.

FIG. 10 is a set of representative FACS plots of viable MV4-11 cells (indicated by arrows) in different treatment groups, and shows therapeutic effect of the combination therapy of 20 ug/ml RPS19 (MIF-I) and 80 nM GILT on MV4-11 after three days in vitro.

FIGS. 11A-11F depict therapeutic effect of GILT and NFκB-inhibitor on MV4-11 and primary AML-FLT3 BMMNC cells. FIG. 11A is a set of representative FACS plots of viable CD44⁺ MV4-11 cells in different treatment groups. Arrows indicate further analyses of Ki67 expression of these viable CD44⁺ blasts. FIG. 11B is a graphical representation of cumulative FACS percentage data of viable CD44⁺ MV4-11 cells. FIG. 11C is a graphical representation of cumulative FACS percentage data of viable Ki67⁺CD44⁺ MV4-11 cells. FIG. 11D is a set of representative FACS plots of viable CD33⁺CD13⁺ primary blasts (Patient #3) in different treatment groups. Arrows indicate further analyses of Ki67 expression of these viable CD33⁺CD13⁺ primary blasts. FIG. 11E is a graphical representation of cumulative FACS percentage data of viable CD33⁺CD13⁺ primary blasts. FIG. 11F is a graphical representation of cumulative FACS percentage data of viable Ki67⁺CD33⁺CD13⁺ primary blasts. *P<0.05, **P<0.01.

FIGS. 12A-12E depict therapeutic effect of NFκB-inhibitor and siRNA knockdown of NFκB2 in bone marrow mononuclear cells (BMMNC). FIG. 12A is a set of graphical representations of mRNA expressions of classical (NFκB1) and non-classical (NFκB2) pathways after different TKI treatment (80 nM). FIG. 12B is a graphical representation of NFκB2 mRNA expressions at 2 days after the siRNA-NFκB2 treatment (50 nM) with or without 80 nM GILT in vitro. The cells were collected for RNA isolation and gene expressions were analyzed by qPCR (N=3). FIGS. 12C, 12D, and 12E are graphical representations of mRNA expressions of MIF, CXCR2 and CXCL5 genes, respectively, after transiently knocking down NFκB2 in MV4-11 cells. *P<0.05, **P<0.01.

FIGS. 13A-13E depict therapeutic effect of the combination of 50 uM NFκB inhibitor and 80 nM GILT in a newly diagnosed AML-FLT3 BMMNC (patient #5). FIG. 13A is a set of representative FACS plots of viable CD33⁺CD13⁺ primary blasts in different treatment groups; Arrows indicate further analyses of Ki67 expression of these viable CD33⁺CD13⁺ primary blasts. FIG. 13B is a graphical representation of cumulative FACS percentage data of viable CD33⁺CD13⁺ primary blasts. FIG. 13C is a graphical representation of cumulative FACS percentage data of viable Ki67⁺CD33⁺CD13⁺ primary blasts. FIG. 13D is a set of images of partial blot films developed for proteomic analyses of cell-free supernatants from GILT-treated primary AML BMMNC cells. The solid arrows indicate the control dots from the manufacturer. The lined, hatched, and dotted arrows indicate the dots of CXCL1, CXL5 and CXCL8 respectively in the film. FIG. 13E is a set of graphical representations of cumulative mean pixel densities (Fold Change) of CXCL1, CXCL5, CXCL8. *P<0.05, **P<0.01,

FIGS. 14A-14D depict therapeutic effect of the combination of 50 uM NFκB inhibitor and 80 nM GILT in a refractory AML-FLT3-ITD BMMNC cells (#7). FIG. 14A is a set of representative phase-bright images showing tumorigenic cluster formation in different experimental groups 3 days after the treatment of different TKI drugs ex vivo. FIG. 14B is a set of representative FACS plots of viable CD117⁺CD13⁺ primary blasts in different treatment groups. Arrows indicate further analyses of Ki67 expression of these viable CD117⁺CD13⁺ primary blasts. FIG. 14C and FIG. 14D are graphical representations of mRNA expressions of the genes of CYCLIN E1 and CD44, respectively, at 3 days after the treatment ex vivo. The cells were collected for RNA isolation and gene expressions were analyzed by qPCR. *P<0.05, **P<0.01.

FIG. 15 is a graphical representation of Cumulative Mean Pixel Densities (Fold Change) of CXCL8, which were acquired from proteome analyses of cell-free supernatants from treated groups of primary AML BMMNC cells. *P<0.05, **P<0.01.

FIGS. 16A-16H depict therapeutic effect of GILT-based combinatory treatments for MV4-11 at 3 days and 7 days after the treatment in vitro (N=4). FIG. 16A is a set of representative FACS plots of viable blast populations in different combination therapies on MV4-11 cells in vitro. The circle indicates viable blasts in a FACS plot. The arrows indicate the dying tendency of blasts which will become positive for viability dye during the next few days. FIG. 16B is a graphical representation of cumulative FACS percentage data of viable MV4-11 cells in different experimental groups at 3 days after the treatment in vitro. *P<0.05, **P<0.01. FIG. 16C is a set of representative FACS plots of Ki67⁺CD33⁺CD44⁺ populations in different combination therapies on MV4-11 cells after their continuous culture in vitro (7 days after the treatment). FIG. 16D is a graphical representation of cumulative cell number count of Ki67⁺CD33⁺CD44⁺ MV4-11 in different experimental groups. *P<0.05, **P<0.01. FIGS. 16E-16H are representative phase-bright images of MV4-11 cells, without treatment and treated with GILT-based combinations as indicated. Scale bar: 100 μm. Arrows indicate tumorigenic clusters in GILT-treated blasts and GILT/STAT4-inhibitor-treated blasts.

FIG. 17A is an illustration of the survival mechanism of CD33⁺CD44⁺pBAD⁺ blasts after TKI treatment. FIG. 17B is an illustration of the intracellular changes of CD33⁺CD44⁺pBAD⁺ blasts after TKI treatment. FIG. 17C is an illustration of the TKI-activated pathways responsible for blast survival and proliferation.

### DETAILED DESCRIPTION

Relapsed or refractory AML patients with mutations in FLT3 gene have poor response to salvage chemotherapy. Even with the new generation of FLT3 inhibitors, the median overall survival is less than one year. Resistance to such inhibitors, primary or secondary, invariably developed along the treatment course suggests that alternative pathways are responsible for the survival of relapsed/refractory leukemic cells. Evolutionarily, duplicated gene pairs (“redundant” elements) in the cellular system can overlap in function to ensure biological robustness for all living beings. However, the concept of treatment-activated compensation systems through “redundant genes” is not well-known to the AML field, and their potential roles in AML relapse have not been examined. Accordingly, the present disclosure provides methods of detecting tyrosine kinase inhibitor (TKI)-resistant AML blasts by determining levels of novel biomarkers CD33, CD44, phosphorylated-BAD (pBAD), and combination thereof in subjects having or at risk of having AML. The present disclosure also provides a combination therapy of TKI and one or more inhibitors targeting a TKI-activated compensatory pathway. The TKI-activated compensatory pathway to be targeted includes a TKI-activated cell survival mechanism, a TYK2-STAT4-PIM2/3 pathway, and a TKI-activated cell proliferation mechanism, a NFκB2-MIF-CXCR2 pathway. Such combination therapies can treat AML more effectively compared to a TKI therapy alone.

A “therapeutically effective amount” is an amount sufficient to effect desired clinical results (i.e., achieve therapeutic efficacy). A therapeutically effective dose can be administered in one or more administrations. “Administering” refers to the physical introduction of a therapeutic agent to a subject in need thereof. Exemplary routes of administration for agents to treat AML include intravenous, intramuscular, subcutaneous, intraperitoneal, spinal or other parenteral routes of administration, for example by injection or infusion. Modes of administration include, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent may be administered via a non-parenteral route, or orally. Other routes include a topical, epidermal or mucosal route of administration, for example, intranasally, vaginally, rectally, sublingually or topically. Administering can also be performed, for example, once, a plurality of times, and/or over one or more extended periods. Therapeutic agents can be constituted in a composition, e.g., a pharmaceutical composition containing the agent and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible.

The present disclosure describes various embodiments related to compositions and methods for management or treatment of cancers, such as AML, gastric cancer, or breast cancer. In the following description, numerous details are set forth in order to provide a thorough understanding of the various embodiments. Before the present methods and compositions are described, it is to be understood that these embodiments are not limited to particular methods or compositions described, as such may, of course, vary. It is also to be understood that the terminology used herein is for the purpose of describing particular embodiments only, and is not intended to be limiting, as the scope of the present embodiments will be limited only by the appended claims. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

A “subject” refers an animal, such as a mammal, including a primate (such as a human, a non-human primate, e.g., a monkey) and a non-primate (such as a mouse). In some aspects of the invention, the subject is a human. In some aspects, the subject is a pediatric subject, such as a neonate, an infant, or a child. In other aspects, the subject is an adult subject.

A “patient” refers to a subject who shows symptoms and/or signs of a disease, is under treatment for disease, has been diagnosed with a disease, and/or is at risk of developing a disease. A “patient” can be human and veterinary subjects. Any reference to subjects in the present disclosure, should be understood to include the possibility that the subject is a “patient” unless clearly dictated otherwise by context. More specifically, the subject in certain aspects is a patient who has a liquid cancer, such as a leukemia.

As used herein, the terms “treating”, “treatment” and the like, shall include the management and care of a subject or patient for the purpose of combating a disease, condition, or disorder and includes the administration of a composition to prevent the onset of the symptoms or complications, alleviate the symptoms or complications, reduce at least one associated sign, symptom, or condition, or eliminate the disease, condition, or disorder.

Treatment also refers to a prophylactic treatment, such as prevention of a disease (e.g., AML) or prevention of at least one sign, symptom, or condition associated with the disease (e.g., relapse of AML). Treatment can also mean prolonging survival as compared to expected survival in the absence of treatment.

As used herein, “⁺” in the context of a biomarker, a cell surface marker, or a cellular molecule refers to a positive status or presence of said marker. As used herein, “-” in the context of a biomarker or a cell surface marker refers to a negative status or absence of said marker. The positive/negative status of an amount of a biomarker, a cell surface marker, or a cellular molecule can be determined by any methods known in the art, including flow cytometry, Western blot, ELISA, and PCR.

In some embodiments, the methods of the present disclosure inhibit a TKI-activated TYK2-STAT2/4-PIM2/3 compensation pathway. TKI-resistant blasts initially express higher CD44 expression with pBAD⁺ relative to non-TKI-resistant blasts. Both the increased expression of CD44 and pBAD in resistant blasts gradually decrease as they survive and regain the capabilities to cluster and grow confluent. Mechanistically, CD44 and its variant isoforms are known to activate different downstream signaling pathways, such as the PI3K/AKT and Src/MAPK, leading to cancer cell invasion and proliferation. Blocking hyaluronic acid, the main CD44 ligand, results in inhibition of BAD phosphorylation. Additionally, mRNA of PIM2, PIM3, and AKT1 are significantly increased in TKI-resistant blasts. After TKI treatment, increased CD44 can result in the activation of PIM2/3 kinase compensation pathways. Thus, TKI-suppressed PIM1 kinase activities are restored to phosphorylate BAD proteins and to prevent their binding to BCL-2, thereby allowing blasts to survive the TKI treatment. In the cellular system, redundant elements can compensate for one another such as in the activation of PIM2 to compensate the lack of PIM1. In hepatocellular carcinoma, the JAK/STAT (STAT3) pathway can regulate CD44⁺ tumor-initiating cells' roles in self-renewing, sphere formation and possible drug resistance. On the other hand, the JAK/STAT (STAT4) pathway may interact with CD44 in AML to indirectly regulate PIM2.

Role of CD44 in Activation of the Kinase Compensation Pathways to Restore the Suppressed Phosphorylation of BAD after TKI Treatment

FIG. 17A is an illustration of the survival mechanism of CD33⁺CD44⁺pBAD⁺ blasts after TKI treatment. Under the condition of no treatment, there is an intracellular homeostatic balance scale (BCL-2 versus BAX) inside the AML blast. Three (3) days after QUIZ or GILT treatment, non-phosphorylated BAD accumulated. Non-phosphorylated BAD tips the scale towards cell death. Over the next few days, the compensation pathways inside TKI-resistant blasts (CD33⁺CD44⁺pBAD⁺ cells) were activated, resulting in phosphorylated BAD (pBAD) and making up for the loss of PIM1 by TKI-suppression (details of intracellular mechanisms in FIG. 17B). FIG. 17B is an illustration of the intracellular changes of CD33⁺CD44⁺pBAD⁺ blasts after TKI treatment. The presence of pBAD tips the scale towards cell survival.

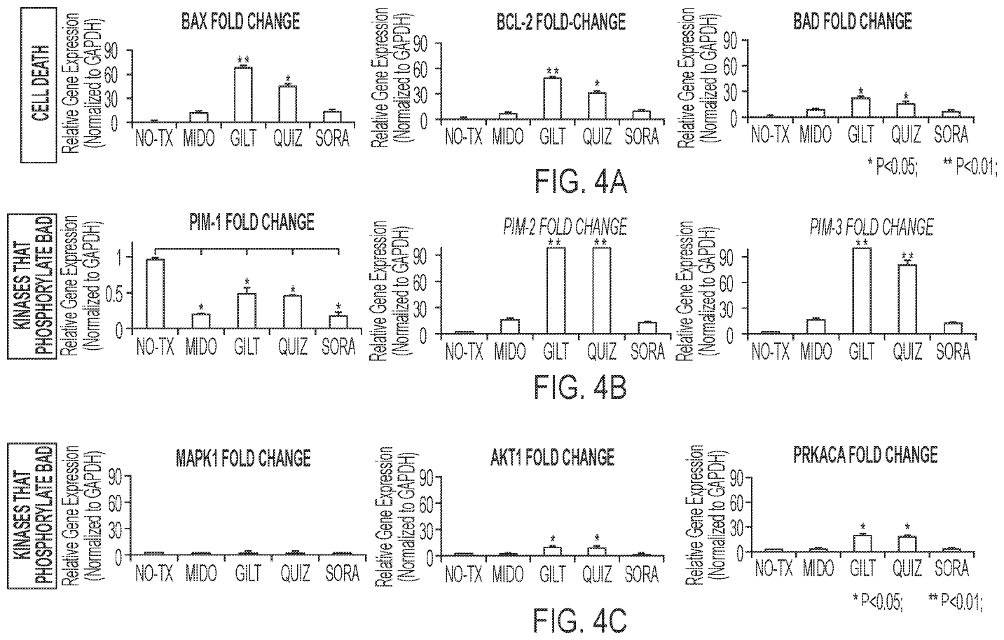

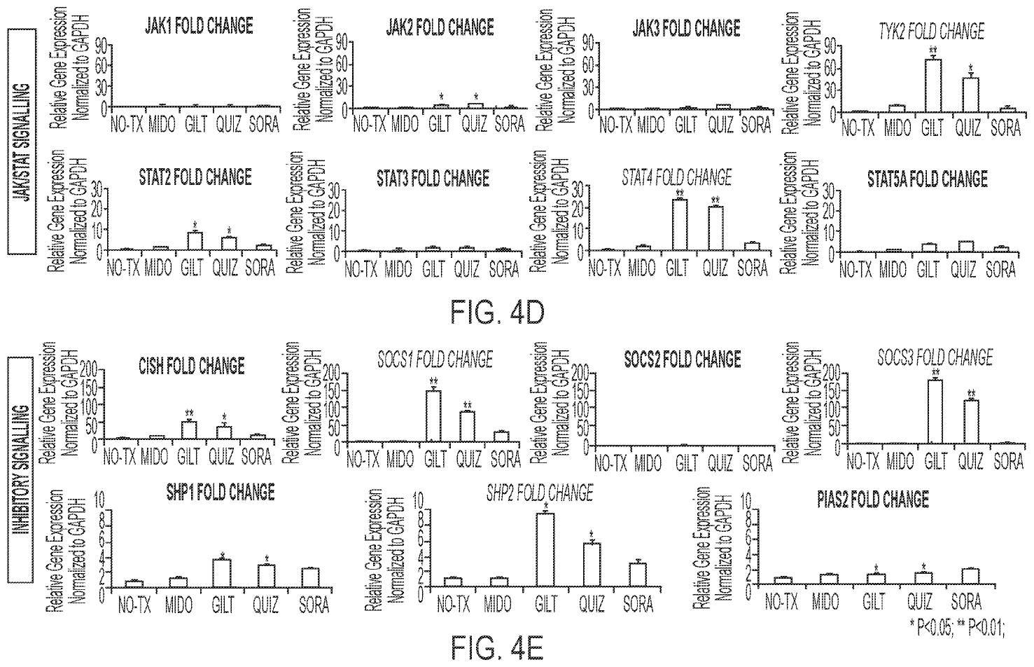

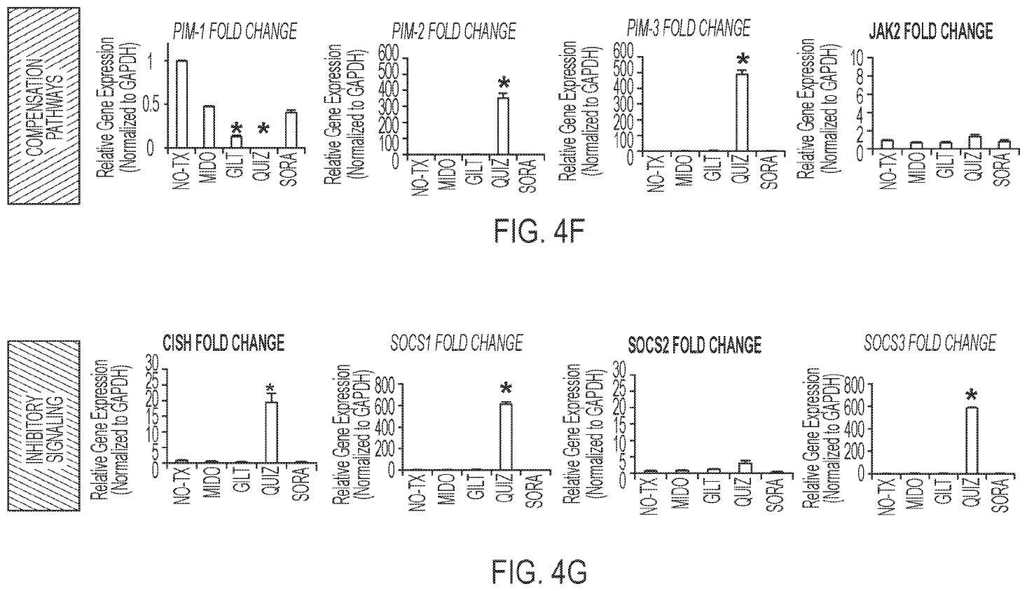

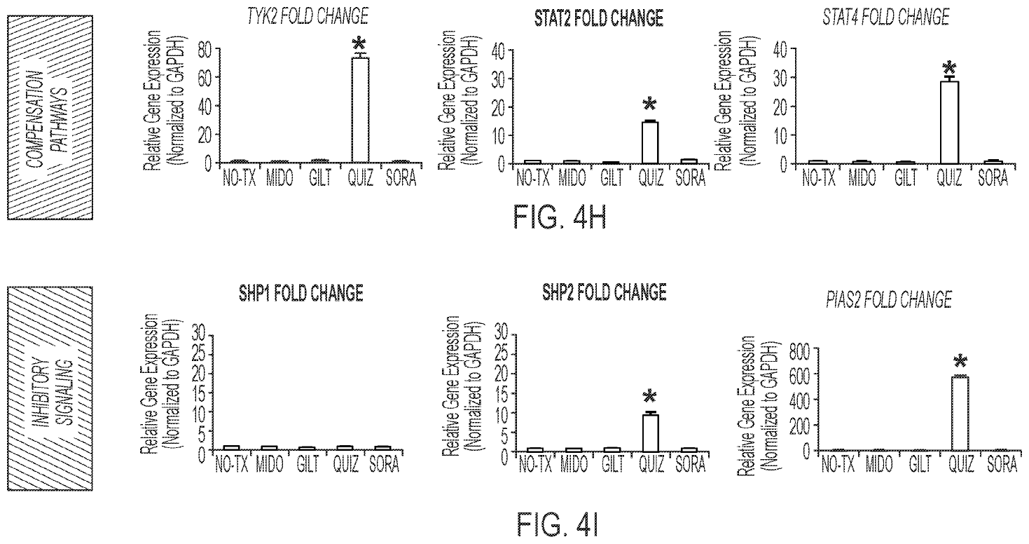

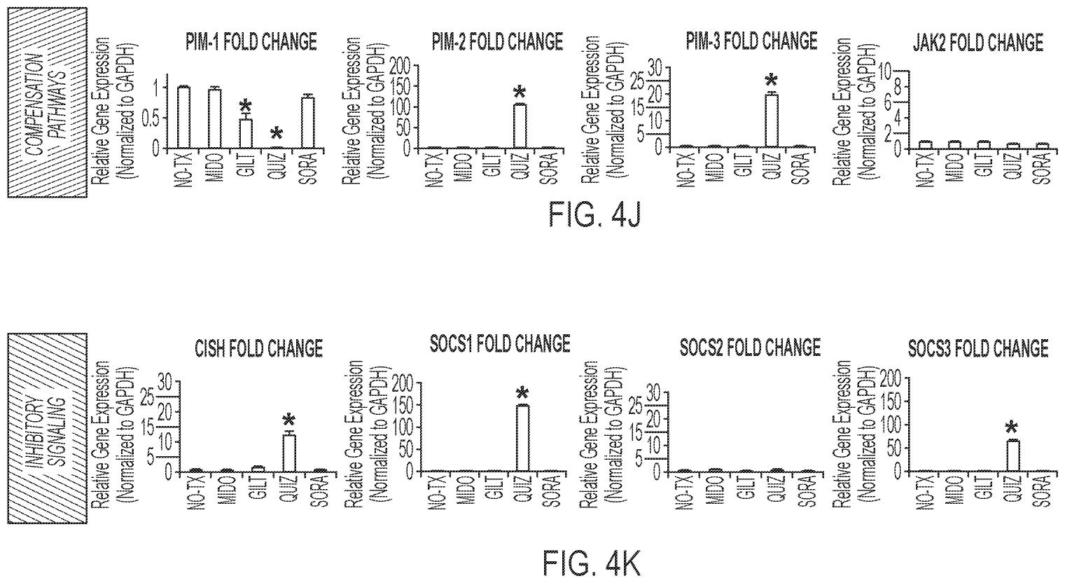

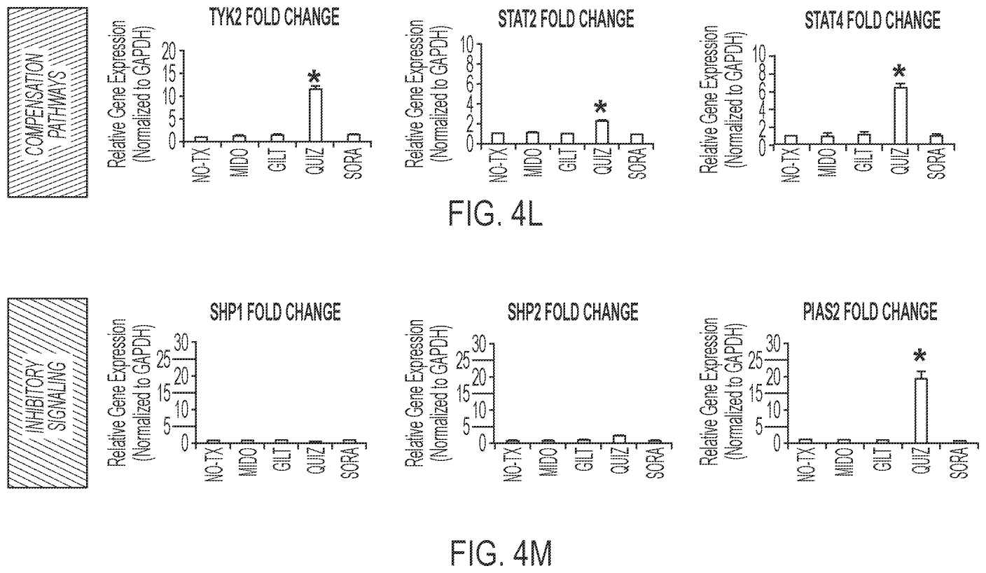

For example, ten (10) days after QUIZ or GILT treatment, tumorigenic clusters formed as observed under the microscope. These tumorigenic clusters were collected for FACS staining and identified as CD33⁺CD44⁺pBAD⁺ cells. In this time interval, mRNA of PIM2, PIM3, TYK2, STAT4, SOCS1, SOCS2, SHP2, and PIAS2 attained their peak fold change values when compared to the non-treatment control (details in FIG. 4E). During the next 2 weeks, many floating tumorigenic clusters were found, and they gradually became confluent as floating single blasts. In this time interval, mRNA of PIM2, PIM3, TYK2, STAT4, SOCS1, SOCS2, SHP2, and PIAS2 were down-regulated to reach intracellular homeostasis.

FIG. 17B schematically depicts intracellular changes of CD33⁺CD44⁺pBAD⁺ blasts after TKI treatment. The TKI treatment blocks the FLT3-related down-stream signaling, causing the inability of the STAT5-PIM1 pathway to phosphorylate BAD. Then, the non-phosphorylated BAD binds to BCL-2 and prevents the anti-apoptotic role of BCL-2, resulting in a high number of blast deaths. Meanwhile, to survive the TKI-treatment, TKI-resistant blasts activate compensation pathways with cytokines released from dead blasts/debris. Then, the activated JAK/STAT signaling pathways or CD44 pathways stimulate the gene expression of alternative kinases such as PIM2/PIM3, the family genes of PIM1, to restore the phosphorylation of BAD. The pBAD loses its function of binding BCL-2, hindering the onset of apoptosis and promoting the TKI-resistant blasts' survival.

To regain the intracellular homeostasis inside TKI-resistant blasts, increased CD33 recruits more SHP1/SHP2 to de-phosphorylate pBAD into a functional BAD. In addition, STAT4 activates the intracellular inhibitory pathways SOCS1/SOCS3 to suppress SHP1/2, CD33, other active kinases, and themselves to prevent over-activated compensation pathways and maintain intracellular homeostasis in TKI-resistant blasts.

CD44 is a surface receptor on a large number of cells (including HSCs and AML blasts) that interacts with ligands like hyaluronic acid. In breast cancer, highly tumorigenic stem cells were found in the CD44⁺ populations. Increasing expression of CD44 (e.g., CD44high) plays an important role in increased adhesion of malignant HSCs to the bone marrow (BM) matrix, which impairs normal differentiation and accumulation of immature blasts in BM niches. This confirms a previous study indicating that CD44 expression levels were higher in patients with myelodysplastic syndrome progressing to AML relative to healthy controls. Examples herein demonstrate that TKI-resistant blasts initially expressed higher CD44 expression with pBAD⁺. The time-course studies showed that both the increased expression of CD44high and pBAD in resistant blasts gradually decreased as they survived and regained the capabilities to cluster and grow confluent. Mechanistically, CD44 and its variant isoforms are known to activate different downstream signaling pathways including the PI3K/AKT, Src/MAPK, leading to cancer cell invasion and proliferation. Blocking hyaluronic acid, the main CD44 ligand, resulted in inhibition of BAD phosphorylation. The qPCR analyses of different kinases disclosed herein also showed significantly increased mRNA of PIM2, PIM3, AKT1, etc. in TKI-resistant blasts. The data shows that after TKI treatment, increased CD44 results in the activation of PIM2/3 kinase compensation pathways. The TKI-suppressed PIM1 kinase activities are restored to phosphorylate BAD proteins and prevent their inhibitory role in BCL-2 (FIG. 17B). In hepatocellular carcinoma, the JAK/STAT (STAT3) pathway has recently been reported to regulate CD44⁺ tumor-initiating cells' roles in self-renewing, sphere formation and possible drug resistance. However, the data herein showed increased expression of STAT4 after the TKI-treatment, and PIM2 was found to be one of STAT4 targeted genes. Thus, the JAK/STAT (STAT4) pathway may interact with CD44 in AML to indirectly regulate PIM2 (FIG. 17B).

CD33 is a well-known marker on the surface of AML blasts and is involved in their proliferation and survival, but the exact role of CD33 and its regulation have not been elucidated. Decreased CD33 expression has been reported on the surface of blasts in children with AML associated with a good prognosis Previously, CD33 was reported to be a myeloid specific inhibitory receptor containing a cytoplasmic immunoreceptor tyrosine-based inhibitory motif (ITIM) with functions in recruiting the phosphatases-SHP-1 and SHP-2. During inflammation, cytokine-induced SOCS3 was reported to not only compete with SHP1/SHP2 by binding to ITIMs of CD33, but also accelerating proteasomal degradation of itself and CD33. In addition, SOCS1/SOCS3 are both target genes of STAT4. In this disclosure, the FACS data showed that increased JAK/STAT (STAT4) pathways and CD33 expression in TKI-resistant blasts gradually decreased the level of control, matching the qPCR data of the significantly increased mRNA of SOCS1, SOCS3, SHP1, SHP2, PIAS2, and then the down-regulation of these mRNA after blast recovery. JAK/STAT (STAT4) pathways are involved in initiating inhibitory pathways to suppress over-reaction after TKI treatment in resistant blasts while CD33 is involved in the recruitment of SHP1/SHP2 for de-phosphorylation of pBAD to balance the survival and cell death (FIG. 17B). The up-down changes of CD33 and CD44 in TKI-resistant blasts was exploited to choose immunotherapies, such as bi-specific monoclonal antibodies, chimeric antigen receptor T cell therapies (CAR-T), or bio-engineered Tumor infiltrating Lymphocytes (TILs) to target CD33 and/or CD44 for the treatment of AML.

In some embodiments, the methods of the present disclosure inhibit a TKI-activated NFκB2-MIF/CXCLs-CXCR2 compensation pathway. After TKI treatment, cytotoxicity-induced injury signals may directly activate the non-canonical NFκB2 (P100/P52) pathway to release more MIF, CXCL5, CXCL8, and other tumor-promoting inflammatory cytokines. In addition to blocking immune cells, MIF acts as an autocrine signal to initiate the survival mechanism through MIF-CD74/CD44 pathways. Meanwhile, MIF-CXCR2, CXCL5-CXCR2 and CXCL8-CXCR2 pathways may be responsible for cell proliferation by activating CDK4/CYCLIN E-based transition from G1 to S phase of the cell cycle progression.

In addition to its role in pBAD-based survival (FIG. 17B), TKI-activated TYK2-STAT4-PIM2/3 may phosphorylate STAT4 (pSTAT4), thereby regulating NFκB2-MIF-CXCR2 and enabling relapse/proliferation of survivor blasts (FIG. 17C). FIG. 17C is an illustration of the TKI-activated pathways responsible for blast survival and proliferation. Accordingly, inhibition of pro-tumor inflammation is also critical to prevent AML relapse.

In some aspects, the present disclosure provides methods of detecting TKI-resistant AML blasts by determining levels of one or more biomarkers CD33, CD44, and phosphorylated-BAD (pBAD) in subjects having or at risk of having AML. In some embodiments, CD33 positive, CD44 positive, and pBAD positive (CD33⁺CD44⁺pBAD⁺) AML blasts are determined to be at an increased risk of developing TKI resistance. In some embodiments, CD33⁺CD44high⁺pBAD⁺ AML blasts are determined to be at an increased risk of developing TKI resistance.

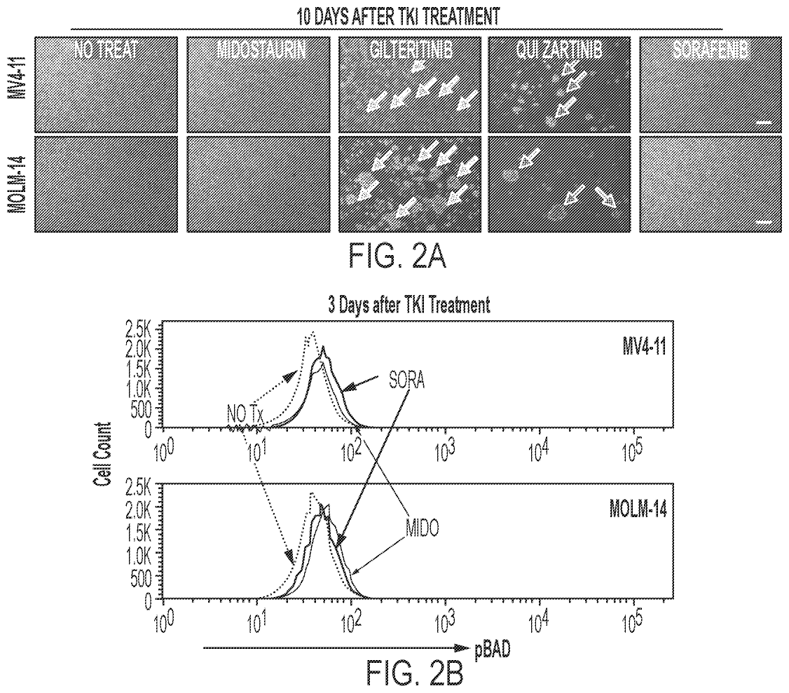

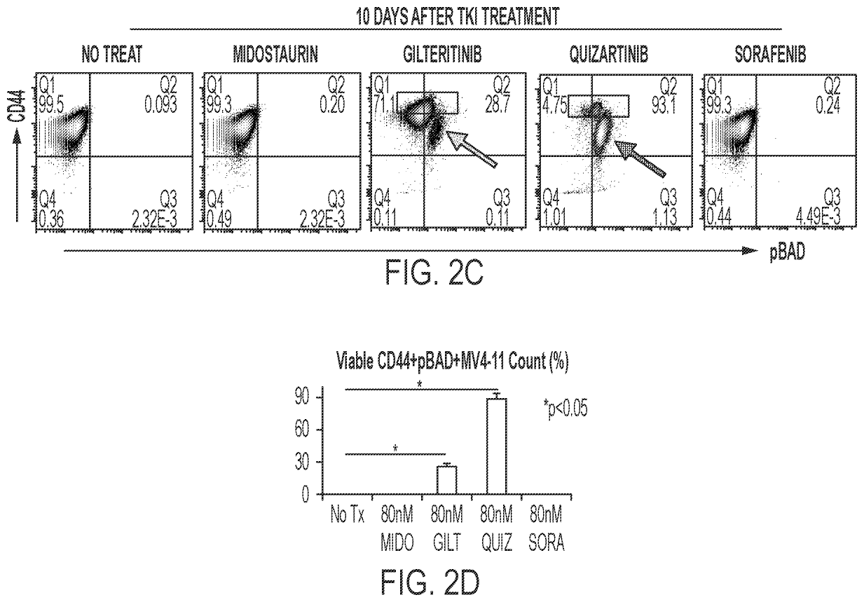

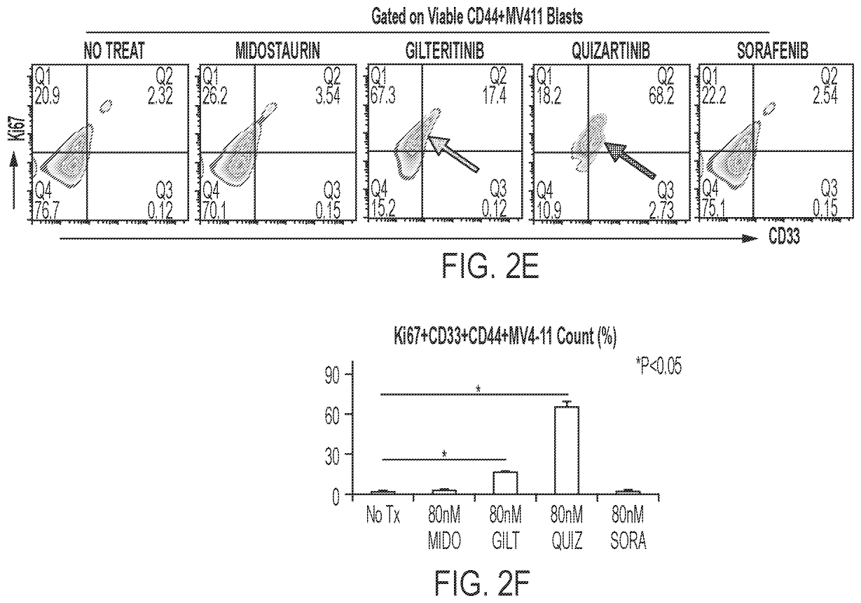

CD33 is also known as Siglec-3, sialic acid binding Ig-like lectin 3, SIGLEC3, SIGLEC-3, gp67, or p67. Human CD33 has a UniProt ID P20138. CD44 is also known as CDW44, CSPG8, ECMR-III, HCELL, HUTCH-I, IN, LHR, MC56, MDU2, MDU3, MIC4, or Pgp1. Human CD44 has a UniProt ID P16070. Bcl-2 associated agonist of cell death (BAD) is also known as BBC2 and BCL2L8. Human BAD has a UniProt ID Q92934. As used herein, “CD44high”, “CD44high⁺”, or “CD44^{lo}” refers to higher expression of CD44 within a CD44⁺ population. For example, boxes in FIG. 2C third and fourth panels indicate CD44high⁺ subpopulations. “CD44low”, “CD44low⁺”, or “CD44^{lo}” refers to lower expression of CD44 within a CD44⁺ population. CD44⁺ cells can be classified into “CD44high” and “CD44low” subpopulations by methods known in the art, such as flow cytometry (e.g., Chaffer et al. 2013 Cell 154:61-74; Ghuwalewala et al. 2016 Stem Cell Res 16:405-417).

Evaluating subjects at an increased risk of developing TKI resistant AML according to the methods of the present disclosure can enable formulation of targeted therapy for such subjects. In some embodiments, the targeted therapy involves TKIs that are FLT3 inhibitors, such as Midostaurin (MIDO), Sorafenib (SORA), Gilteritinib (GILT), and QUIZ (QUIZ).

Disclosed herein are methods of treating acute myeloid leukemia (AML) in a subject, comprising administering a therapeutically effective amount of a TKI and a therapeutically effective amount of an inhibitor of a TKI-activated compensation pathway or an inhibitor of the intracellular homeostasis required for leukemic relapse after treatment. to the subject. In some embodiments, the subject has TKI-resistant AML or is at risk of developing TKI-resistant AML. The TKI of the combination therapy can be one or more of MIDO, SORA, GILT, and QUIZ. The inhibitor of a TKI-activated compensation pathway can be an inhibitor of a JAK-STAT pathway, a TYK2-STAT4-PIM2/PIM3 pathway, and/or a NFκB2(P100/P52)-MIF-CXCR2 pathway. In some embodiments, the inhibitor can be one or more of a STAT4 inhibitor, a BCL2 inhibitor, a CD44 inhibitor, a CXCR2 inhibitor, and an NFκB inhibitor. In some specific embodiments, the methods include administering a therapeutically effective amount of a TKI, a therapeutically effective amount of a BCL2 inhibitor, and a therapeutically effective amount of a NFκB inhibitor to the subject. In other embodiments, the methods include administering a therapeutically effective amount of a TKI, a therapeutically effective amount of a STAT4 inhibitor, a therapeutically effective amount of a BCL2 inhibitor, and a therapeutically effective amount of a NFκB inhibitor to the subject. In some embodiments, the STAT4 inhibitor is (R)-Lisofylline. In some embodiments, the BCL2 inhibitor is Venetoclax. In some embodiments, the NFκB inhibitor is MG-132 or BAY 11-7082. In some embodiments, the CD44 inhibitor is a urokinase-derived peptide, Angstrom6. In some embodiments, the CXCR2 inhibitor may be a potent, selective and non-peptide antagonist, such as SB225002. Embodiments of a combination of a TKI and an inhibitor of a TKI-activated compensation pathway are set forth in Table 1 and Table 2.

In some embodiments, administering a combination of a TKI (e.g., GILT) and an inhibitor of a TKI-activated compensatory pathway (such as a STAT4 inhibitor, a BCL2 inhibitor, a CD44 inhibitor, a CXCR2 inhibitor, and an NFκB inhibitor) synergistically increases cell death of AML tumor cells as compared to administering a TKI alone or an inhibitor of a TKI-activated pathway alone. In some embodiments, a combination of a TKI (e.g., GILT) and an inhibitor of a TKI-activated compensatory pathway (e.g., a STAT4 inhibitor, a BCL2 inhibitor, a CD44 inhibitor, a CXCR2 inhibitor, and an NFκB inhibitor) is administered to a patient after the detection of specific AML tumor cells, such as Ki67⁺CD33⁺CD44⁺pBAD⁺ cells, as compared to administering a TKI alone or an inhibitor of a TKI-activated pathway alone.

Administering a combination of a therapeutically effective amount of a TKI and a therapeutically effective amount of an inhibitor of a TKI-activated compensatory pathway can increase cell death of AML tumor cells by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more compared to administering a TKI alone or an inhibitor of a TKI-activated pathway alone.

In some embodiments, administering a combination of a therapeutically effective amount of a TKI and a therapeutically effective amount of an inhibitor of TKI-activated compensatory pathway (e.g., a STAT4 inhibitor, a BCL2 inhibitor, a CD44 inhibitor, a CXCR2 inhibitor, and an NFκB inhibitor) synergistically reduces or inhibits AML blast relapse compared to administering a TKI alone or an inhibitor of a TKI-activated pathway alone. Administering a combination of a therapeutically effective amount of a TKI and a therapeutically effective amount of an inhibitor of a TKI-activated compensatory pathway can reduce AML blast relapse by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100% compared to administering a TKI alone or an inhibitor of a TKI-activated pathway alone.

Exemplary combinations of a TKI and one or more inhibitors of a TKI-activated compensatory pathway are indicated with “X” in Table 2 below. Each inhibitor of a TKI-activated compensatory pathway includes its species and variants, such as drugs that possess the inhibitory activity. Combinations are not limited to those listed herein. Any other agents and combinations of agents may be used for methods according to the present disclosure.

TABLE 2
Therapeutic Agent Combinations

| | | Inhibitor of TKI-activated compensatory pathway | | | | |
|---|---|---|---|---|---|---|
| | | STAT4 | BCL2 | | | NFκB inhibitor |
| | | inhibitor (e.g., | inhibitor | CD44 inhibitor | CXCR2 | (e.g., BAY11- |
| | | (R)- | (e.g., | (e.g., | inhibitor (e.g., | 7082, BAY11- |
| | | Lisofylline) | Venetoclax) | Angstrom6) | SB225002) | 7821) |
| TKI | Gilteritinib | X | | | | |
| | (GILT) | | X | | | |
| | | | | X | | |
| | | | | | X | |
| | | | | | | X |
| | | X | X | | | |
| | | X | | X | | |
| | | X | | | X | |
| | | X | | | | X |
| | | | X | X | | |
| | | | X | | X | |
| | | | X | | | X |
| | | | | X | X | |
| | | | | X | | X |
| | | | | | X | X |
| | | X | X | X | | |
| | | X | X | | X | |
| | | X | X | | | X |
| | | X | | X | X | |
| | | X | | X | | X |
| | | X | | | X | X |
| | | | X | X | X | |
| | | | X | X | | X |
| | | | X | | X | X |
| | | | | X | X | X |
| | | X | X | X | X | |
| | | X | X | X | | X |
| | | X | X | | X | X |
| | | X | | X | X | X |
| | | | X | X | X | X |
| | | X | X | X | X | X |
| | Quizartinib | X | | | | |
| | (QUIZ) | | X | | | |
| | | | | X | | |
| | | | | | X | |
| | | | | | | X |
| | | X | X | | | |
| | | X | | X | | |
| | | X | | | X | |
| | | X | | | | X |
| | | | X | X | | |
| | | | X | | X | |
| | | | X | | | X |
| | | | | X | X | |
| | | | | X | | X |
| | | | | | X | X |
| | | X | X | X | | |
| | | X | X | | X | |
| | | X | X | | | X |
| | | X | | X | X | |
| | | X | | X | | X |
| | | X | | | X | X |
| | | | X | X | X | |
| | | | X | X | | X |
| | | | X | | X | X |
| | | | | X | X | X |
| | | X | X | X | X | |
| | | X | X | X | | X |
| | | X | X | | X | X |
| | | X | | X | X | X |
| | | | X | X | X | X |
| | | X | X | X | X | X |
| | Midostaurin | X | | | | |
| | (MIDO) | | X | | | |
| | | | | X | | |
| | | | | | X | |
| | | | | | | X |
| | | X | X | | | |
| | | X | | X | | |
| | | X | | | X | |
| | | X | | | | X |
| | | | X | X | | |
| | | | X | | X | |
| | | | X | | | X |
| | | | | X | X | |
| | | | | X | | X |
| | | | | | X | X |
| | | X | X | X | | |
| | | X | X | | X | |
| | | X | X | | | X |
| | | X | | X | X | |
| | | X | | X | | X |
| | | X | | | X | X |
| | | | X | X | X | |
| | | | X | X | | X |
| | | | X | | X | X |
| | | | | X | X | X |
| | | X | X | X | X | |
| | | X | X | X | | X |
| | | X | X | | X | X |
| | | X | | X | X | X |
| | | | X | X | X | X |
| | | X | X | X | X | X |
| | Sorafinib | X | | | | |
| | (SORA) | | X | | | |
| | | | | X | | |
| | | | | | X | |
| | | | | | | X |
| | | X | X | | | |
| | | X | | X | | |
| | | X | | | X | |
| | | X | | | | X |
| | | | X | X | | |
| | | | X | | X | |
| | | | X | | | X |
| | | | | X | X | |
| | | | | X | | X |
| | | | | | X | X |
| | | X | X | X | | |
| | | X | X | | X | |
| | | X | X | | | X |
| | | X | | X | X | |
| | | X | | X | | X |
| | | X | | | X | X |
| | | | X | X | X | |
| | | | X | X | | X |
| | | | X | | X | X |
| | | | | X | X | X |
| | | X | X | X | X | |
| | | X | X | X | | X |
| | | X | X | | X | X |
| | | X | | X | X | X |
| | | | X | X | X | X |
| | | X | X | X | X | X |

### EXAMPLES

The following examples are offered by way of illustration and not by way of limitation.

### Example 1: Biomarkers for TKI-Resistant AML Blasts

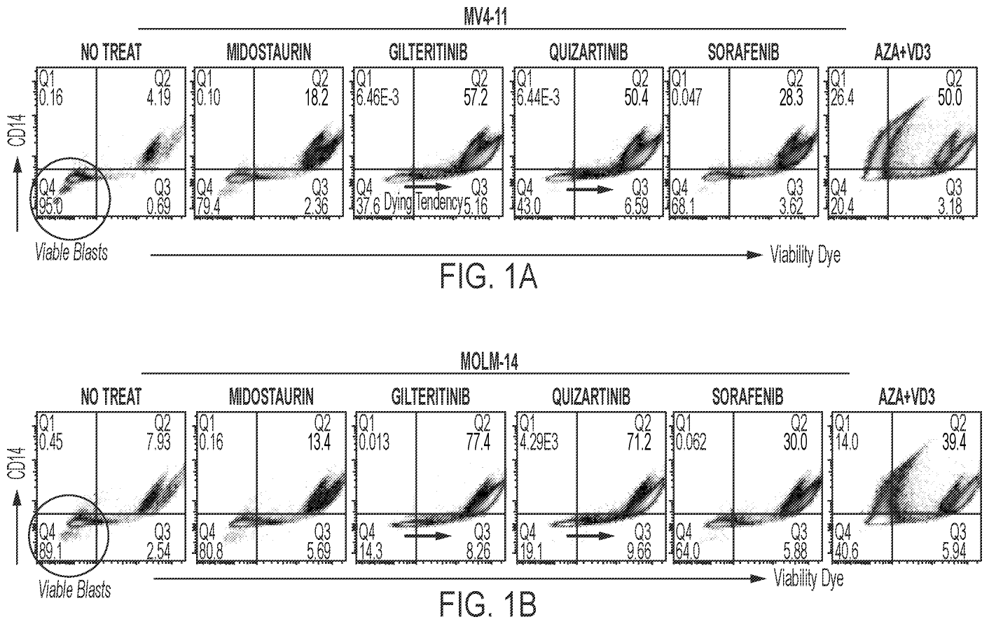

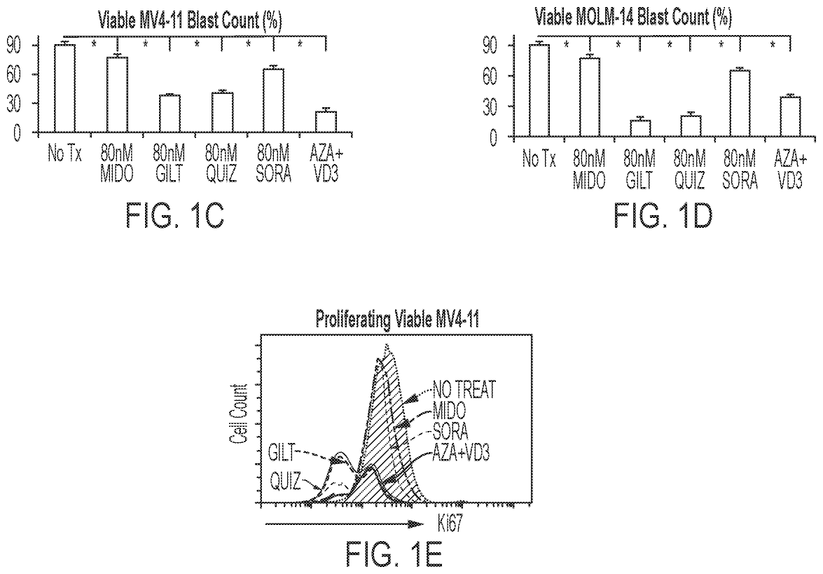

The AML cells were analyzed at different time points after exposure to TKI (3 days, 10 days, 20 days, and 28 days). First, MV4-11 and MOLM-14 cells were subject to in vitro TKI-treatment using MIDO, SORA, GILT, and QUIZ. After 3 days, FACS of the surface markers and intracellular proteins, including CD14, CD33, CD44, and Ki67, were performed. FIGS. 1A-1E depicts therapeutic effect of different TKIs on MV4-11 and MOLM-14 cells in vitro (N=6). FIG. 1A shows representative FACS (fluorescence-activated single cell sorting) plots of different TKI inhibitors and 5-Azacitidine (AZA) and 1α,25-Dihydroxyvitamin D3 (VD3) on MV4-11 cells after 3 days of in vitro treatment. FIG. 1B shows representative FACS plots of different TKI inhibitors and AZA/VD3 on MOLM-14 cells after 3 days of in vitro treatment. The circles indicate the group of viable blasts located in a FACS plot and green arrows indicate the dying tendency of blasts which will become positive for viability dye during the next few days. FIGS. 1C and 1D are graphical representations of the cumulative FACS percentage data of viable MV4-11 and MOLM-14 cells. *P<0.05. FIG. 1E is a representative FACS plot of Ki67 expression in different treatment groups after 3 days of in vitro treatment. Black arrows indicate the expression curve of Ki67, showing treated groups with less expression of Ki67 when compared to the non-treatment control.

All four TKIs significantly reduced viable MV4-11 and MOLM-14 blasts (FIG. 1A-D) when compared to the non-treatment controls. These TKIs mainly worked by cytotoxicity instead of by inducing maturation of the blasts, in contrast to large populations of viable CD14′ cells differentiated by the treatment of AZA and VD3 (FACS plots of AZA and VD3, FIG. 1A, B). Among the TKIs, GILT and QUIZ as the second-generation therapies were more effective than the first-generation FLT3 inhibitors-MIDO and SORA, as expected (FIG. 1A-D). Furthermore, all four TKIs significantly reduced proliferation of viable blasts (Ki67 Histogram, FIG. 1E) when compared to the non-treatment controls. Morphologically, newly formed blast clusters were observed in MIDO and SORA treated cultures, but not in GILT and QUIZ treated cells. Residual blasts underwent a continuous trend of apoptosis days after treatment (Green arrows, FIG. 1A, B).

To follow the cell fate of TKI-treated MV4-11 and MOLM-14, culturing of blasts were continued by performing medium change every 2-3 days. Ten (10) days after TKI treatment, it was found that both MIDO and SORA treated MV4-11 and MOLM-14 grew confluent in cultures and had normal morphology like the non-treatment controls (Images of MIDO, SORA, FIG. 2A). In contrast, there were many newly formed tumorigenic clusters among numerous floating dead cells in GILT treated cultures, and few newly formed tumorigenic clusters in QUIZ treated cultures (arrows, Images of GILT, QUIZ, FIG. 2A). The BAD protein is a pro-apoptotic member of the BCL-2 gene family, which is involved in initiating apoptosis. Dephosphorylated BAD is pro-apoptotic by binding BCL-2 and inactivating BCL-2, while phosphorylated BAD (pBAD) is anti-apoptotic by leaving BCL-2 free to inhibit BAX-triggered apoptosis. The FACS data showed the significant increase in pBAD expressions at early stages (3 days after the treatment) in both MIDO and SORA treated MV4-11 and MOLM-14 (pBAD Histogram, FIG. 2B). Ten days after TKI treatment, FACS analysis revealed significantly increased populations of viable CD44⁺pBAD⁺ cells in GILT (28.7%) and QUIZ (93.1%) treated cultures, in contrast to control (0.093%), MIDO (0.2%) and SORA (0.24%) experimental groups (arrows, FIG. 2C, D). There were also significantly increased populations of viable Ki67⁺CD33⁺CD44⁺ cells in GILT (17.4%) and QUIZ (68.2%) treated cultures, in contrast to control (2.32%), MIDO (2.54%), and SORA (2.54%) treated cultures (arrows, FIG. 2E, F).

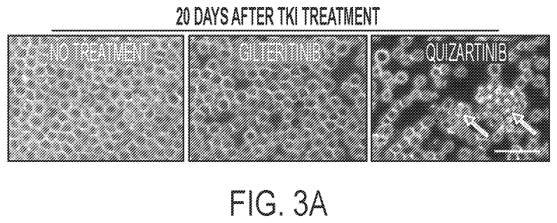

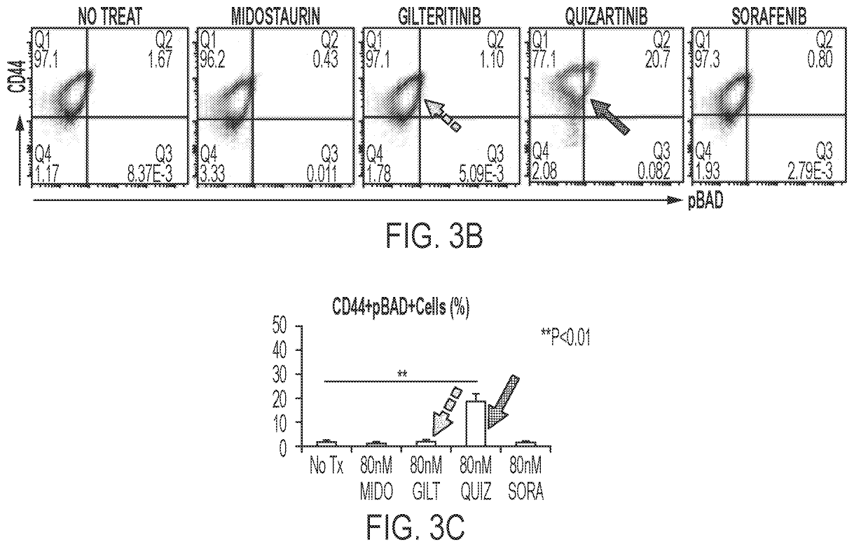

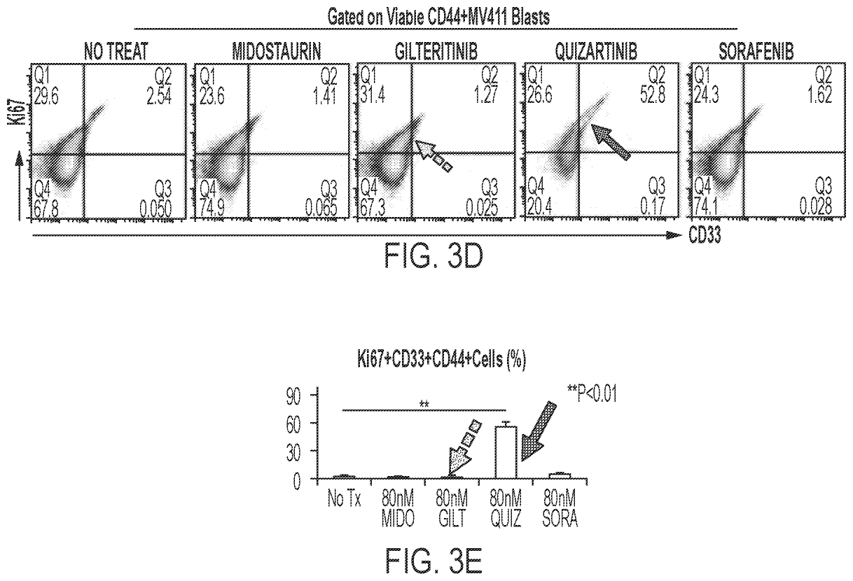

Twenty days after TKI treatment, GILT-treated MV4-11 grew confluent without tumorigenic clusters in cultures and had normal morphology like non-treatment controls (FIG. 3A). However, there were still many tumorigenic clusters among the floating viable cells in Quiz treated cultures (arrows, Image of QUIZ, FIG. 3A). FACS analyses revealed that previous significantly increased populations of viable CD44⁺pBAD⁺ cells in GILT (28.7%) (arrow, FIG. 2C), had decreased to the level (1.1%) similar to control (1.67%), MIDO (0.43%) and SORA (0.8%) treated cultures (arrow, FIG. 3B, C). The data also showed that previous significantly increased populations of viable Ki67*CD33⁺CD44⁺ cells in GILT treated cells (17.4%) (arrow, FIG. 2E) had decreased to the level (1.27%) similar to control (2.54%), MIDO (1.41%) and SORA (1.62%) treated cultures (FIG. 3D, E). Similar findings were found in the QUIZ-treated group with previous significantly increased populations of viable CD44⁺pBAD⁺ cells in QUIZ (93.1%) (arrow, FIG. 2C), decreasing to 20.7% (arrows, FIG. 3B, C). Also, prior significantly increased populations of viable Ki67⁺CD33⁺CD44⁺ cells in QUIZ (68.2%) (red arrow, FIG. 2E), had decreased to 52.8% (arrow, FIG. 3D, E), consistent with their current recovery status with many tumorigenic clusters. In summary, the data shows that TKI-resistant MV4-11 are CD33⁺CD44⁺pBAD⁺ cells and undergo mitosis as shown by the expression of Ki67+. Further time-course studies showed that increased expression of CD44, pBAD, and CD33 in resistant blasts gradually decreased when they survived and regained the capabilities to cluster and grow confluent.

### Example 2: TKI-Activated Cell Survival Mechanism: A TYK2-STAT4-PIM2/3 Pathway

Pro-survival gene expression in TKIs treated leukemic cells were investigated at different time points after exposure to TKI (3 days, 10 days, 20 days, and 28 days). The data shows intracellular homeostatic change within TKI-resistant blasts (Stage 1 Activation, Stage 2 Cool down).

MV4-11 cells (3 days after TKI treatment) were harvested and analyzed by reverse transcription quantitative polymerase chain reaction (qPCR). The qPCR data of four genetic aspects including cell death, kinases specialized in phosphorylation of BAD, JAK/STAT signaling pathway, and intrinsic inhibitory pathways were compared. The data showed that cell death related BAX mRNA, BCL-2 mRNA and BAD mRNA in GILT-treated MV4-11 cells when compared to those in the control non-treatment cells were significantly increased by 68-fold, 49-fold, and 23-fold, respectively (FIG. 4A). These findings are consistent with the FACS data showing a high number of dead cells (Viability Dye⁺) in GILT-treated MV4-11 cells. Similar findings in BAX mRNA, BCL-2 mRNA and BAD mRNA also occurred in QUIZ-treated MV4-11 cells when compared to those in the control non-treatment cells, with significant increases of 44-fold, 31-fold, and 16-fold, respectively (FIG. 4A). Next, to investigate which kinases are responsible for the phosphorylation of BAD (pBAD) after TKI treatment, screenings of those kinase candidates reported to phosphorylate BAD under physiological conditions were conducted. In contrast to significant decreases in the PIM1 mRNA from TKI-treated cells when compared to those in the control non-treatment cells, PIM2 mRNA and PIM3 mRNA in GILT-treated MV4-11 cells were significantly increased compared to the control (FIG. 4B), consistent with a previous report of PIM2 related TKI-resistance. For example, the qPCR data showed that the increased folds of the PIM2 and PIM3 mRNA in GILT-treated MV4-11 cells are the highest among all screened kinase genes (203-fold up in PIM2 and 134-fold up in PIM3 versus 10-fold up in AKT1 and 15-fold up in PRKACA, FIG. 4B). Similar expression changes in PIM1 mRNA, PIM2 mRNA and PIM3 mRNA occurred in QUIZ-treated MV4-11 cells when compared to those in the control non-treatment cells with a significant decrease of 0.5-fold, increase of 120-fold, and increase of 81-fold, respectively (FIG. 4B and FIG. 4C). Elevated gene expressions of both PIM2 and PIM3 have been found in adult AML patients associated with poor survival. Also, overexpressed PIM3 has been found to promote AML cell proliferation and protect against spontaneous apoptosis by phosphorylating BAD.

The Janus kinase (JAK)/signal transducer and activator of transcription (STAT) pathway consists of a group of cytokine receptors and transmembrane proteins that recognize specific cytokines, and is critical in healthy blood formation and the immune response. However, dysregulation of the JAK/STAT signaling pathway provides a survival advantage to tumorigenic cells by transmitting anti-apoptotic and proliferative signals in different cancers, including blood malignancies. In AML FLT3, a large increase in STAT5 from the JAK/STAT pathway has been linked to the sustained activation of FLT3-signaling pathways, resulting in AML cell survival and proliferation. With this in mind, a screening of all gene expressions in the JAK/STAT pathway was performed. The data displayed a significant increase of TYK2 mRNA and JAK2 mRNA in GILT-treated MV4-11 cells when compared to those in the control non-treatment cells by 71-fold and 6-fold, respectively (FIG. 4D). Similar patterns in TYK2 mRNA and JAK2 mRNA expression changes occurred in Quiz-treated MV4-11 cells when compared to those in the control non-treatment cells with significant increases of 46-fold and 6-fold, respectively (FIG. 4D). In addition, the increased folds of both STAT4 mRNA and STAT2 mRNA in GILT-treated MV4-11 cells when compared to those in the control non-treatment cells were the highest among all screened STAT genes (23-fold up in STAT4 and 9-fold up in STAT2 versus 4-fold up in STAT5A and 2 fold up in STAT3, FIG. 4D). Similar findings in STAT4 mRNA and STAT2 mRNA also occurred in Quiz-treated MV4-11 cells when compared to those in the control non-treatment cells as they were significantly increased by 19-fold and 6-fold, respectively (FIG. 4D). This significant increase in STAT4 is consistent with a recent report that there are existing STAT4-mediated pathways (TKI-resistant) and STAT5-mediated pathways (TKI-sensitive) capable of regulating PIM2's role in the phosphorylation of BAD in chronic myeloid leukemia (CML). STAT4 activation involves STAT2 recruitment.

Next, the intrinsic homeostasis of the blast, particularly how cytokine signal transduction and kinase activation are switching off, was investigated. Signaling through the JAK-STAT pathway is tightly controlled by a number of endogenous feedback inhibitors including suppressors of cytokine signaling (SOCS), protein inhibitors of activated STATs (PIASs), and protein tyrosine phosphatases (PTPs) like SHP1/2. Accordingly, a screening of gene expression in the SOCS family was performed. The data showed that SOCS1 mRNA and SOCS3 mRNA in GILT-treated MV4-11 cells when compared to those in the control non-treatment cells were significantly increased by 153-fold and 182-fold, respectively (FIG. 4E). Similar findings in SOCS1 mRNA and SOCS3 mRNA occurred in Quiz-treated MV4-11 cells when compared to those in the control non-treatment cells since the mRNA were significantly increased by 88-fold and 120-fold, respectively (FIG. 4E). In addition, the genes encoding phosphatases like the SHP1 mRNA and the SHP2 mRNA in GILT-treated MV4-11 cells when compared to those in the control non-treatment cells were significantly increased (9-fold up in SHP2, about 4-fold up in SHP1, about 2-fold up in PIAS2, FIG. 4E). Protein inhibitor of activated STAT2 (PIAS2), a member of the PIAS family, has been reported to be a negative regulator of Stat4. Similar increased folds of SHP1 mRNA and SHP2 mRNA also occurred in QUIZ-treated MV4-11 cells when compared to those in the control non-treatment cells (5-fold up in SHP2, about 3-fold up in SHP1, and about 2-fold up in PIAS2, FIG. 4E).

Ten (10) days after TKI treatment, mRNA of PIM2 (355-fold up), PIM3 (494-fold up), TYK2 (73-fold up), STAT2 (14-fold up), STAT4 (28-fold up, SOCS1 (615-fold up), SOCS3 (590-fold up), SHP2 (9-fold up), and PIAS2 (570-fold up) attained their peak fold change values in QUIZ-resistant blasts when compared to the non-treatment control (FIGS. 4F-4I). Increase in mRNA of these genes were similarly observed twenty (20) days after TKI treatment (FIGS. 4J-4M). At this moment, most QUIZ-resistant blasts that survived were CD44⁺pBAD⁺ (93.1%, red arrow, FIG. 2C), and they were starting to form a few tumorigenic clusters (arrows, FIG. 2A). In sum, the qPCR and FACS data demonstrate that TYK2-STAT2/4-PIM2/3 pathways play vital roles in the survival and tumorigenic proliferation of TKI-resistant blasts. Meanwhile, the up-regulated counterpart inhibitory pathways suggest that there is an intrinsic homeostatic system to suppress over-activation of JAK/STAT pathways and to support the TKI-resistant blasts' recovery.

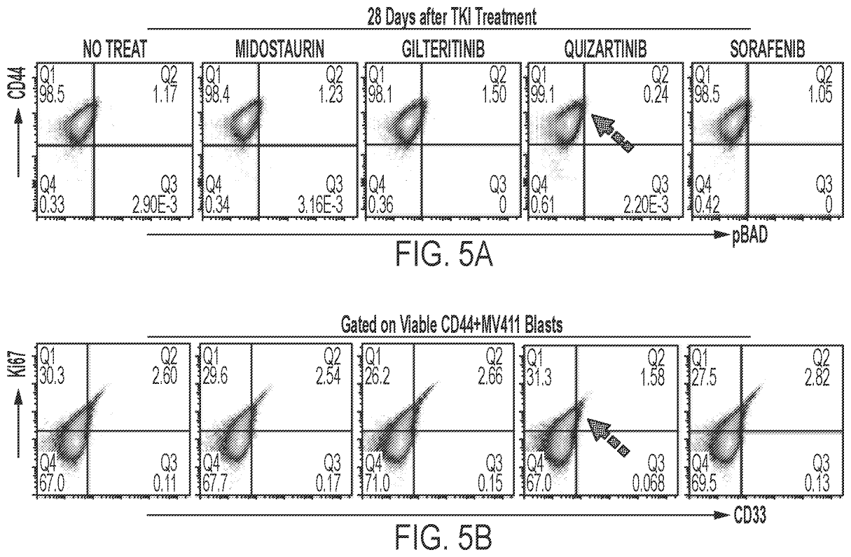

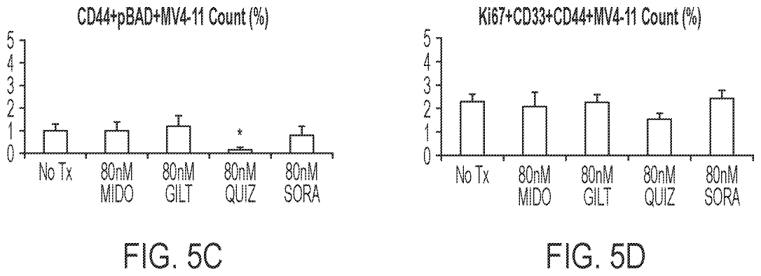

TKI-resistant blasts at late stages after TKI treatment were investigated. Twenty-eight days after TKI treatment, QUIZ-treated MV4-11 grew confluent without tumorigenic clusters in cultures and had normal morphology like non-treatment controls (not shown but similar to Image of GILT, FIG. 3A). FACS analyses revealed that previous significantly increased populations of viable CD44⁺pBAD⁺ cells in QUIZ (20.7%) (arrow, FIG. 3B) had decreased to the level (0.24%) similar to the control (1.17%), MIDO (1.23%), and SORA (1.05%) treated cultures (arrow, FIGS. 5A, 5C). Also, previous significantly increased populations of viable Ki67⁺CD33⁺CD44⁺ cells in QUIZ (52.8%) (arrow, FIG. 3D) had decreased to the level (1.58%) similar to the control (2.6%), MIDO (2.54%) and SORA (2.82%) treated cultures (arrow, FIG. 5B, D).

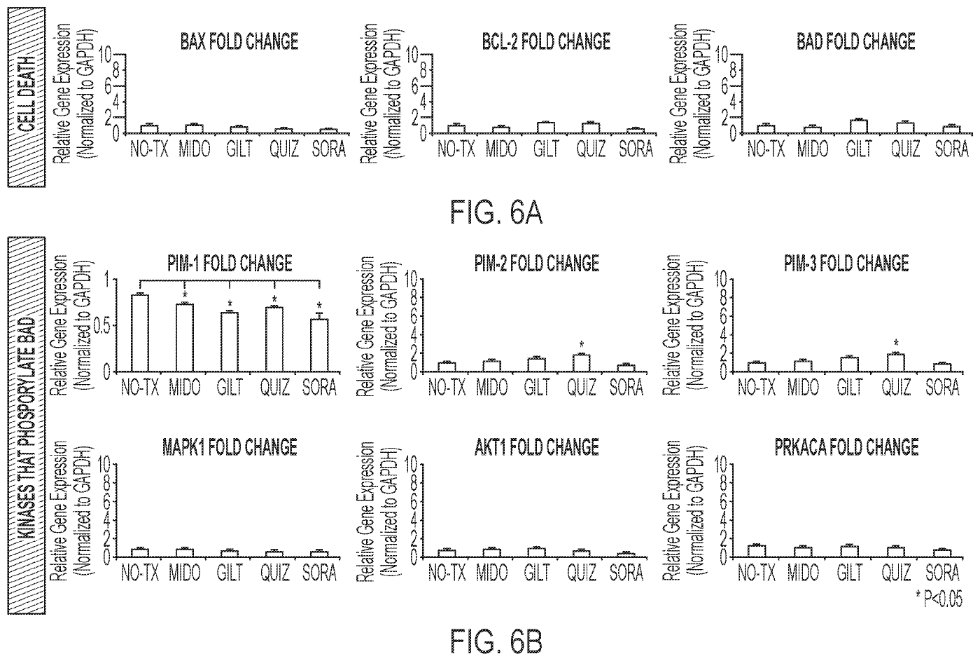

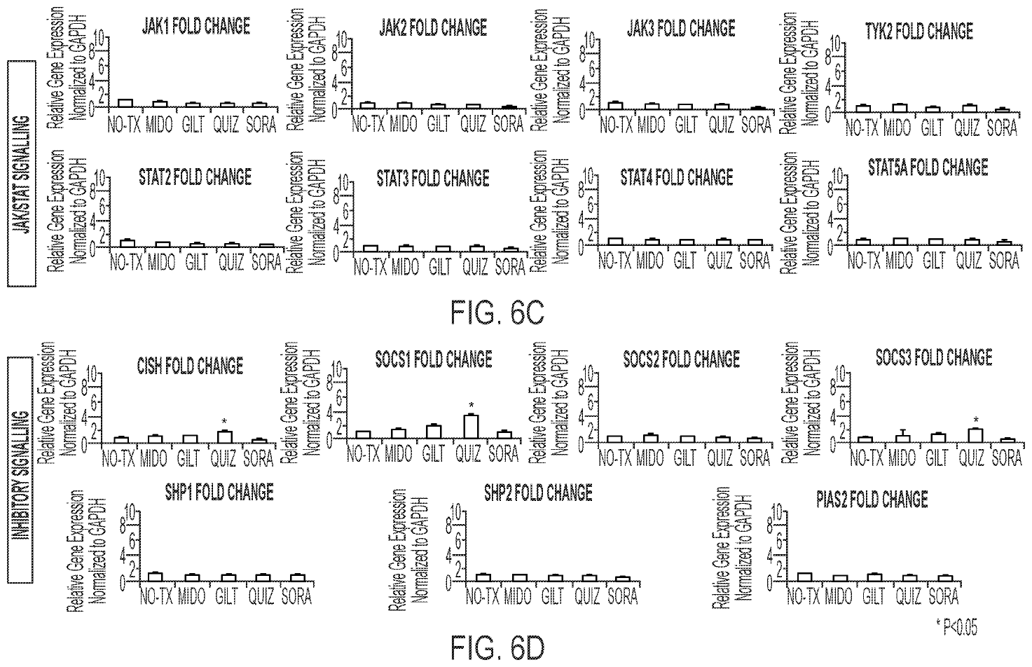

After 28 days, all four TKI-treated blasts relapsed in vitro. Whether their gene expressions recovered was also evaluated. The same sets of qPCR primers were used on the RNA isolated from the blasts 28 days after the treatment to compare four genetic aspects including cell death, kinases specialized in phosphorylation of BAD, JAK/STAT signaling pathway, and intrinsic inhibitory pathways. The data showed that there was no significant difference in BAX mRNA, BCL-2 mRNA and BAD mRNA in TKI-treated MV4-11 cells when compared to those in the control non-treatment cells (FIG. 6A). Among the kinase genes, all TKI-treated MV4-11 still had significant reductions of PIM1 mRNA (FIG. 6B). Meanwhile, only the QUIZ-treated MV4-11 still had a significant increase of about 2-fold of PIM2 and PIM3 mRNA (FIG. 6B). There was no significant difference in JAK/STAT in TKI-treated MV4-11 cells when compared to those in the control non-treatment cells (FIG. 6C). Among the inhibitory pathways, only the QUIZ-treated MV4-11 still had a significant increase of CISH mRNA, SOCS1 mRNA, and SOCS3 mRNA by about 2-fold, 4-fold, and 2-fold when compared to those in the control non-treatment cells (FIG. 6D). In summary, the data of TKI-resistant blasts at the late stage are consistent with the confluent relapse of blasts in vitro because the late-stage TKI-resistant blasts have been more similar to the non-treatment cells than the early-stage TKI-resistant blasts have been.

The time-course studies shown in Examples 1-2 demonstrated that after TKI treatment, the relapsed blasts with tumorigenic capabilities are CD33⁺CD44⁺pBAD⁺ cells, and they are undergoing intracellular homeostasis to regenerate leukemic growth (FIG. 17A).

### Example 3: Release of Macrophage Migration Inhibitory Factor (MIF) and Upregulation of CXCR2 by TKI-Treated Blasts

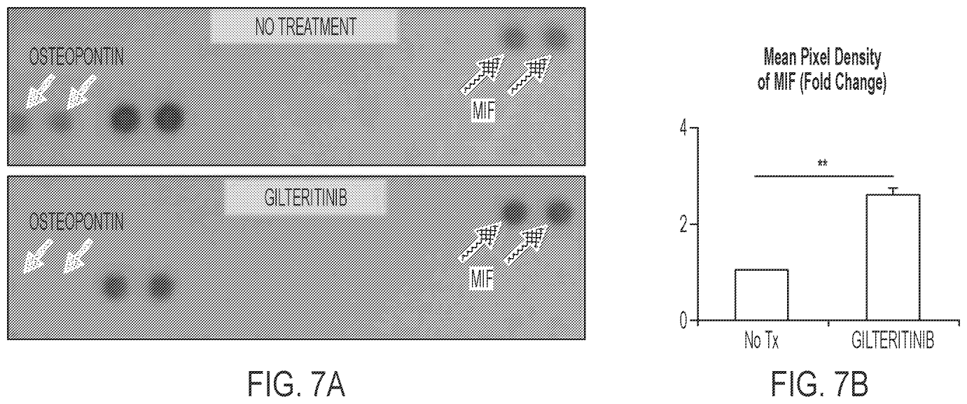

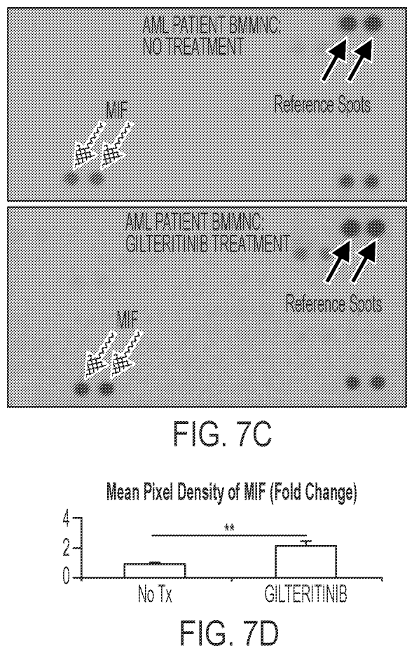

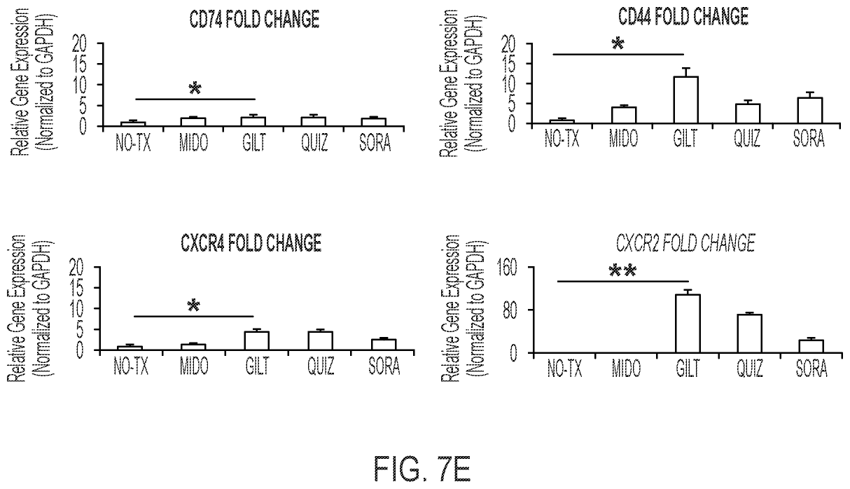

To identify the protein(s) responsible for initiation of the survival mechanism and subsequent proliferation of AML blasts, human cytokine proteome profile arrays of cell-free supernatants from GILT-treated MV4-11's cultures were performed. Notably, the data revealed that macrophage migration inhibition factor (MIF) was significantly increased by 2.6-fold in the supernatant from MV4-11 after the treatment when compared to the non-treatment control (arrows, FIG. 7A; mean pixel density shown in FIG. 7B). Under the no treatment (No Tx) condition, MV4-11 releases numerous cytokines such as MIF in their supernatant; however, one dose of GILT-treatment eliminated release of a variety of cytokines such as osteopontin (arrows, FIG. 7A), a protein closely associated with adverse prognosis in AML patients. Next, bone marrow mononuclear cells (BMMNC) from AML patients were treated for 3 days ex vivo. In the GILT-treated AML samples (Newly diagnosed Patient #1 and Refractory Patient #2, Table 4), MIF release was also found to be significantly increased by 2.1-fold in the supernatant when compared to the non-treated control (FIGS. 7C, 7D).

To investigate which transmembrane proteins interact with MIF to transduce the signaling after different TKI treatments, gene expressions of all encoded MIF receptors were compared. The qPCR data showed significantly increased CXCR2 mRNA (109-fold) in GILT-treated MV4-11 cells (FIG. 7E). The increased fold change of CXCR2 mRNA was the highest among all screened MIF receptors (FIG. 7E). CXCR1 mRNA was also found to have a high increase (about 90-fold up, not shown). The data is consistent with a recent report that high CXCR2 expression correlates with poor prognosis in AML patients. In summary, the data demonstrates that: 1) both an AML cell line and primary AML BMMNC release more MIF after TKI treatment; and 2) TKI activates significant up-regulation of CXCR2 gene expression in MV4-11 cells.

### Example 4: MIF Promotes the Proliferation of MV4-11 Through MIF/CXCLs-CXCR2 Pathways and Survival of a Group of CD44High+ Blasts after TKI Treatment

A significant increase in MIF expression has been related to angiogenesis, cell cycle initiation and tumor metastasis in solid tumors; however, the exact role of MIF in AML is not yet fully understood. To examine the role of MIF in the proliferation of AML blasts, different doses of recombinant human MIF peptides were added to the cell cultures of MV4-11 (AML blast cell line). The FACS data showed that 5 ng/ml MIF, the lowest dose, can induce MV4-11's proliferation by expressing a greater percentage of Ki67⁺ (FIG. 8A). To investigate how MIF affects immune cells, MIF was tested on RAW264.7 (a macrophage cell line). Five (5) ng/ml MIF effectively suppressed the RAW264.7's proliferation (FIG. 8B). Furthermore, the qPCR data also showed that exogenous recombinant MIF can effectively induce the gene expressions of chemokine (C-X-C motif) ligand (CXCL) 1, CXCL5, CXCL8 (IL-8), Cyclin-dependent kinase 4 (CDK4), and CYCLIN E1 in MV4-11 cells (FIGS. 8F-8G). The findings are consistent with a previous report that primary AML blasts constitutively express MIF, stimulating bone marrow mesenchymal cells to release CXCL8 (IL-8), a ligand that also binds to CXCR2, to sustain blasts' proliferation. In addition, CXCL1/5/8 were routinely and highly released (a median level >1000 pg/mL) in the supernatants of culturing naïve AML patient cells ex vivo. The data demonstrates that: 1) MIF induces the proliferation of MV4-11; 2) MIF induces the gene expression of CXCR2, a major receptor for different cytokines; 3) MIF induces the gene expression of key AML-promoting cytokines; 4) MIF induces the gene expression of key regulators important for cell cycle G1-S phase progression; and 5) MIF suppresses the proliferation of macrophages which might transiently block immune cells to avoid further damage. The data suggests that the MIF/CXCLs-CXCR2 pathways are activated by TKI-treatment to initiate the tumor-promoting inflammation and support the proliferation of surviving blasts.

To investigate whether MIF/CXCLs-CXCR2 pathways could be activated by TKI-treatment in viable blasts to support their survival and continuous proliferation, a serial coculture was conducted to examine whether the supplementation of exogenous MIF at early (simultaneous) or late stage (sequential) of TKI treatment could rescue the GIL-treated MV4-11 in vitro. CD44, a multifunctional cell surface adhesion receptor, was identified as a key regulator of quiescent AML leukemic stem cells (LSCs) which are highly microenvironment dependent. In the sequential coculture (detailed procedures in the method), the supplementation of MIF to GILT-treated MV4-11 cells significantly increased a group of the viable quiescent CD44⁺ cell population (FIG. 8C, plots). The percentage of this CD44⁺ population was found to significantly increase from 3.66% in untreated control to 17.7% in GILT only, 26.3% in GILT+50 ng/mL MIF and 25.2% in GILT+200 ng/mL MIF (FIGS. 8C-8E). In the simultaneous coculture, another group of CD44High⁺ cells was also found which included both Ki67⁺ and Ki67⁻ subsets (FIG. 8F). This group of CD44High⁺ cells could be visualized in the flow cytometry histogram (FIG. 8G). Meanwhile, the qPCR data revealed that the supplementation of MIF to GILT-treated MV4-11 cells significantly increased the gene expression of CXCR2 (bar chart, FIG. 8G). CD44high⁺ stem-like cells have been previously found to be highly tumorigenic, treatment-resistant and responsible for metastatic progression in breast cancer. Recently, a combined genetic and functional approach to trace the origins of AML relapse demonstrated that therapy-resistant cells were already present at diagnosis and evolved at relapse from either rare LSCs or blasts with stemness transcriptional signatures. The current findings suggest that the significantly increased MIF after TKI treatment might support a group of preexisting LSCs-like blasts with inducible CD44high⁺ expression to survive TKI-treatment and result in the relapse of FLT3mut AML.

### Example 5: TKI-Activated Cell Proliferation Mechanism: NFκB-MIF-CXCR2 Pathway

To investigate what other pathways that transduce MIF signaling, gene expressions of all encoded MIF receptors were compared. The qPCR data showed significantly increased CXCR2 mRNA (109-fold) in GILT-treated MV4-11 cells (FIG. 9A). The increased fold of CXCR2 mRNA was the highest among all screened MIF receptors (FIG. 9A). Furthermore, exogenous recombinant MIF effectively induced CXCR2 expression in MV4-11 blasts in vitro (FIG. 9B), suggesting MIF-CXCR2 signaling might be the proliferative pathway in TKI-resistant blasts. Another question is to identify the transcription factor controlling the gene expression of MIF. Nuclear factor kappa-light-chain enhancer of activated B cells (NFκB) family of transcription factors is known to regulate the expression of both cytokines and their receptors. There are two NFκB activation pathways including NFκB1 (p50-p65, canonical) and NFκB2 (p52-RelB, non-canonical). To examine which NFκB pathway is responsible for TKI resistance, gene expression of NFκB family was compared. The qPCR data showed significantly increased NFκB2 mRNA (110-fold) in GILT-treated MV4-11 when compared to the control (FIG. 9C). The increased fold of NFκB2 was the highest among all NFκB members (not shown). NFκB activation has been reported to be regulated by JAK/STAT signaling pathway in immune cells. Also, as a member of “rapid-acting” transcription factors e.g., c-Jun and STAT, NFκB family is first responder to harmful cellular stimuli. Pro-tumorigenic inflammation has been considered as a potential therapeutic target for the treatment of solid tumors.

### Example 6: Targeting the MIF-CXCR2 Pathway in the Treatment of AML

Whether targeting the MIF-CXCR2 signaling pathway would effectively treat AML blasts were investigated. First, the combination of GILT with ribosomal protein S19 (RPS19) on MV4-11 blasts was tested in vitro. RPS19, a MIF inhibitor, has been reported to suppress cisplatin-activated MIF-CD74-NFκB signaling pathway in acute kidney injury. The data showed that there was no synergistic anti-leukemic effect of the combination of 80 nM GILT and 20 ug/ml RPS19 (FIG. 10). The combination of 80 nM GILT with 50 uM SB225002, a CXCR2-inhibitor (CXCR2-I) on MV4-11 blasts in vitro were examined. The data demonstrated that the combination of GILT plus CXCR2-I significantly reduced the percentage of viable CD44⁺ MV4-11 blasts (0.73% versus 12.2% in GILT only and 92.7% in non-treated control, FIG. 11A/upper plots), and Ki67⁺ CD44⁺ blasts (1.66% versus 12.2% in GILT only, 5.7% in CXCR2-I only, and 83.1% in non-treated control, FIG. 11A/lower plots and 11B/11C bar charts). The anti-leukemic effect of combining GILT with CXCR2-I on primary BMMNC ex vivo were examined. The data again showed that GILT with CXCR2-I significantly reduced the percentage of viable CD33⁺ CD13⁺ primary blasts (1.75% versus 7.14% in GILT only, 3.11% in CXCR2-I only, and 24.9% in non-treated control, FIG. 11D/upper plots), and Ki67⁺CD13⁺ blasts (0% versus 60% in GILT only, 14.3% in CXCR2-I only, and 81.3% in non-treated control, FIG. 11D/lower plots and 11E/11F bar charts (Patient #2 and #3, Table 4). However, primary AML BMMNC from two patients did not respond well to the treatment (Patient #1 and 4, Table 4). The data is consistent with a previous report that inhibition of the CXCL8-CXCR2 axis moderately reduces clone proliferation of MDS/AML cell lines and patient samples. There may be alternative cytokine pathways compensating for the defect of MIF-CXCR2 pathways.

### Example 7: NFκB2 is Responsible for the Activation of MIF/CXCLs-CXCR2 Pathways in Surviving TKI-Resistant Blasts

To abolish the leukemic effect of different cytokines and their dynamic receptors, the transcription factor controlling their gene expressions in TKI-resistant blasts was investigated. Nuclear factor kappa-light-chain enhancer of activated B cells (NFκB), a family of transcription factors, is known to play a key molecular link between inflammation and oncogenic initiation/progression by regulating the expression of different tumor-promoting inflammatory cytokines and their receptors. Also, the NFκB binding region has been described in the MIF promoter to augment MIF transcription. There are two NFκB activation pathways including NFκB1 (p50-p65, canonical) and NFκB2 (p52-RelB, non-canonical). To examine which NFκB pathway is responsible for TKI resistance, gene expression of the NFκB family was compared. The qPCR data showed significantly increased NFκB2 mRNA (110-fold) in GILT-treated MV4-11 when compared to the non-treated control (NO-TX) (right bar chart, FIG. 12A). The increased fold of NFκB2 was much higher than NFκB1 (left bar chart, FIG. 12A). Next, the siRNA knockdown of NFκB2 in vitro was performed and then the genetic changes after the reduction of NFκB2 mRNA were examined (FIGS. 12B-12E). The siRNA knockdown of NFκB2 significantly reduced the NFκB2 mRNA by approximately 3-fold (siRNA-NFκB2 versus non-treated control, FIG. 12B) and by 2-fold in combination with GILT (GILT⁺ siRNA-NFκB2 versus GILT only, FIG. 12B). Transient knockdown of NFκB2 does not affect the gene expression of NFκB1 (data not shown). Furthermore, the data showed that transient silencing of NFκB2 significantly reduced the MIF mRNA by 1.5-fold (siRNA-NFκB2 versus non-treated control, FIG. 12C) and GILT-activated gene expressions of CXCR2 and CXCL5 by 2-fold and 2.6-fold, respectively (GILT⁺siRNA-NFκB2 versus GILT only, FIGS. 12D, E). In summary, the data suggest that NFκB2-MIF/CXCLs-CXCR2 pathways might be responsible for the initiation of tumor-promoting inflammation to support the survival and cell proliferation in TKI-resistant AML blasts.

### Example 8: GILT Combined with NFκB Inhibitor (NFκB-I) Effectively Treated Primary Blasts from Both New Diagnosed and Refractory AML Patients Ex Vivo

The combination of 80 nM GILT and 50 uM BAY11-7821 (a NFκB-I) was tested as a novel therapeutic approach for AML. The data showed that GILT⁺ NFκB-I synergistically reduced the percentage of viable CD33⁺ CD13⁺ primary blasts (0.035% versus 35.8% in GILT only, 0.14% in NFκB-I only, and 51.4% in non-treated control, FIG. 13A/upper plots), and Ki67⁺CD33⁺ proliferating blasts (0% versus 73.3% in GILT only, 25% in NFκB-I only, and 78.9% in non-treated control, FIG. 13A/lower plots and 13B/13C bar charts) (Newly diagnosed AML, Patient #5, and also repeatable in Patient #6, Table 4). Remarkably, the combination of GILT+NFκB-I completely eliminated expression of the tumor-promoting inflammatory cytokines CXCL1, CXCL5 and CXCL8 in the supernatant of treated AML patient specimens when compared to non-treated controls (FIG. 13D/blot films and 13E bar charts).

Next, the anti-leukemic effect of the combination was examined on primary BMMNC from refractory AML patients (Patient #7, Table 4). The data showed that GILT⁺ NFκB-I not only significantly inhibit the tumorigenic clustering in primary BMMNC (images, FIG. 14A), but also significantly reduce the viability of CD117⁺CD13⁺ blasts (FIG. 14B/upper plots) and suppress the proliferation of Ki67⁺CD33⁺CD117⁺CD13⁺ primary blasts (FIG. 14B/lower plots). Mechanistically, the qPCR data showed the combination of GILT⁺ NFκB-I significantly reduced the gene expression of CYCLIN E1, a molecule responsible for the S-phase of the cell cycle (FIG. 14C), and the gene expression of CD44, an adhesion molecule for tumorigenic clustering (FIG. 14D). Lastly, the combinatory treatment was found to eliminate CXCL8 (IL-8) effectively in the supernatant of treated AML patient specimens when compared to GILT only (FIG. 15). In summary, the ex vivo data demonstrated that targeting NFκB2-MIF/CXCLs-CXCR2 pathways can effectively suppress the release of tumor-promoting inflammatory cytokines and their mediated survival and proliferation of primary blasts from both newly diagnosed and refractory AML patients ex vivo.

### Example 9: TKI-Based Combination Therapies for AML Relapse In Vitro

TKI combined with inhibitors targeting TKI-activated compensation systems were proved effectively to treat refractory AML ex vivo. FLT3, the most frequently mutated gene in AML patients, is often associated with poorer overall survival with an increased risk of relapse. However, the anti-leukemic effects of the latest FLT3 inhibitors as monotherapy or in combination with standard treatments are constrained by a short duration of response and a high rate of relapse. Accordingly, whether targeting the compensation pathways along with administration of FLT3 inhibitors, e.g., GILT, prevents relapse. A list of the in vitro trials of GILT in combination with inhibitors (commercially available) targeting new pathways on MV4-11 is provided in Table 1.

| | | | | | | | | | GILT+ |
|---|---|---|---|---|---|---|---|---|---|
| | | | | | | | | GILT+ | BAY11-7082 + |
| | NO TREAT | | GILT+ | GILT+ | GILT+ | GILT+ | GILT+ | BAYII-7082 + | Venetoclax + |
| | (CONTROL) | GILT+ | Lisofylline | Venetoclax | Angstrom6 | SB225002 | BAY11-7082 | Venetoclax | Lisofylline |
| Mechanism | N/A | TKI | TKI+ | TKI+ | TKI+ | TKI+ | TKI− | TKI+ | TKI+ |
| | | | STAT4-1 | BCL2-1 | CD44-1 | CXCR2-1 | NFKB-1 | NFKB-1 + | NFKB-1 + |
| | | | | | | | | BCL-2 | BCL2-1 + |
| | | | | | | | | | STAT4-1 |
| Dosing | N/A | 80 nM | 80 nM + | 80 nM + | 80 nMb + | 80 nM + | 80 nM + | 80 nM + 50 | 80 nM + 50 μM + |
| | | | 10 μM | 100 nM | 100 nM | 50 μM | 50 μM | μM + 100 nM | 100 nM + 10 μM |
| Synergistic | N/A | N/A | YES | YES | NO | YES | YES | YES | YES |
| Effect | | | | | | | | | |
| MV4-11 relapse | YES | YES | YES | NO | YES | NO | NO | NO | NO |
| in vitro (2 | | | | | | | | | |
| weeks) | | | | | | | | | |

A more comprehensive, nonlimiting exemplary combination therapies are set forth in Table 2. Table 1 illustrates examples of new combination therapies targeting NFκB2-MIF-CXCR2 pathways. Primary AML blasts have been found to constitutively express MIF, which stimulates bone marrow mesenchymal cells to release interleukin-8 (CXCL8, IL-8, a ligand binding to CXCR2) to sustain blasts' proliferation. Thus, targeting the receptor CXCR2 prevents leukemic relapse via its ligands MIF and IL-8.

Effect of combining a TKI with pathway driven small molecule inhibitors to suppress the relapse of TKI-resistant MV4-11 in vitro was tested. The detailed description of small molecule inhibitors including doses and catalogs are set forth in Materials and Methods.

FACS analyses 3 days after combination treatment revealed significantly decreased populations of viable blasts in GILT⁺ BCL2 inhibitor (2.8%), GILT⁺ PIM inhibitor (18.6%), and GILT⁺ STAT4 inhibitor (18.7%) treated cultures, in contrast to GILT-only (26.9%) treated cultures (FIGS. 16A, 161B). FACS analyses 7 days after combination treatment revealed that there were also significantly reduced viable Ki67⁺CD33⁺CD44^{m} cells in GILT⁺ BCL2 inhibitor (6 cells) and GILT⁺ STAT4 inhibitor (40 cells) treated cultures, in contrast to GILT-only (180 cells) treated cultures (FIGS. 16C, 16D). Consistent with FACS results, there were newly formed tumorigenic clusters in GILT⁺ STAT4 inhibitor treated cultures, but these clusters appear later and were fewer and smaller than those of non-treatment controls (FIG. 16E) and GILT-only treated cultures at 7 days after the treatment (FIGS. 16F, 16G). In addition, the culture media of both GILT⁺ BCL2 inhibitor and GILT⁺ STAT4 inhibitor treated cultures were much lighter in terms of population density than those of control and GILT-only experimental groups. Finally, in GILT⁺ BCL2 inhibitor treated cultures, most floating cells were dead, consistent with FACS results (Image, FIG. 16H; FACS plot, FIG. 16C), and there were no newly formed tumorigenic clusters 2 weeks after the treatment. The data suggest that combinatory therapies combining a TKI (GILT) with pathway driven small molecule inhibitors like the STAT4 inhibitor ((R)-Lisofylline) or the BCL-2 inhibitor (Venetoclax) can be a novel therapeutic approach to treat refractory AML.

In summary, provided herein are novel molecular and genetic phenotypes of TKI-resistant AML blasts along with their responses to surviving the TKI treatment and their maintaining of intrinsic homeostasis after blast relapse. Also, provided herein is a novel therapeutic approach in combining a TKI with a protein inhibitor targeting a specific JAK-STAT pathway that was more efficacious than the TKI-only treatment. The experimental approach, up-down phenomena of biomarkers, survival mechanisms, and intracellular homeostatic concept in treatment-resistant leukemic blasts provided herein may be applicable to other refractory malignancies as well.

### Materials and Methods

Human Samples. AML bone marrow (BM) mononuclear cells (BMMNC) (Patients #1-7, Table 4) were obtained from the City of Hope National Medical Center (COHNMC). All donor patients signed an informed consent form. Sample acquisition was approved by the Institutional Review Boards at the LLUMC and the COHNMC in accordance with an assurance filed with and approved by the Department of Health and Human Services, and it met all requirements of the Declaration of Helsinki.

Cell Lines and Cell Culture. MV4-11 (ATCC CRL-9591) and MOLM-14 (DSMZ ACC-777) are human derived AML blast cell lines with FLT3-ITD. The AML cells (either MV4-11 or primary AML BMMNC) and RAW264.7 were cultured in RPMI-1640 medium (Hyclone, Thermo Scientific), supplemented with 10% heat-inactivated fetal bovine serum (FBS, HyClone) and 1% penicillin/streptomycin. Cells were grown at 37° C. in a humidified atmosphere containing 5% CO2.

Small Molecular Inhibitors and in vitro & ex vivo Treatment. The list of inhibitors, abbreviations, manufacturers, and catalogue # are found in Table 3. MIDO and SORA are first-generation FLT3 inhibitors, and GILT and QUIZ are second-generation inhibitors. As single agents to treat blasts in vitro, a single dose of 80 nM of MIDO, GILT, QUIZ, or SORA were added to 1 ml of 1×10⁶ cells for each experimental group in 24-well plates. The dose of 80 nM for the four TKIs was selected based on their dose-dependent cytotoxicity in previous reports. The 100 nM MIDO-treatment caused a 60% reduction of viable MV4-11. Both GILT and QUIZ were reported to start to suppress the tumorigenic clustering and c-Kit at the dose of 80 nM. 100 nM SORA can significantly induce apoptosis and cell cycle arrest in MV4-11 after 72-hour treatment in vitro. The combination of 5 μM 5-Azacitidine (AZA) and 80 nM 1α,25-Dihydroxyvitamin D3 (VD3) was added to the experimental group similar to the previous report. As combination agents to treat blasts in vitro, one dose of 80 nM GILT with one dose of either 100 nM Venetoclax (BCL2 inhibitor), 10 μM (R)-Lisofylline (STAT4 inhibitor), 100 nM AZD1208 (PIM inhibitor), 500 nM PF-06826647 (TYK2 inhibitor), or 500 nM AT9283 (JAK2/3 inhibitor) were added to 1 ml of 1×10⁶ cells for each experimental group in 24 well plates. Three days after the one dose treatment, cells were then collected for either analyses by FACS and qPCR, or re-plated into 24 well plates for continued culture. At different time points, re-plated cells were collected for analyses by FACS and qPCR. Medium change was performed every 2-3 days.

For MIF in vitro experiments, different doses of MIF were added to 1 ml of 1×10⁶ MV4-11 cells or RAW264.7 cells for each experimental group in 24-well plates. Two days after the one dose treatment, cells were then collected for either analyses by FACS and qPCR. As combination agents to treat blasts in vitro or ex vivo, one dose of 80 nM GILT with one dose of different inhibitors were added to 1 ml of 1×10⁶ MV4-11 cells or 1 ml of 0.5-1×10⁶ primary AML BMMNC for each experimental group in 24-well plates. Dose-dependent experiments of CXCR2 inhibitor and NFκB inhibitor with or without 80 nM GILT were performed. It was determined that the current dose for either 50 uM CXCR2 inhibitor or 50 uM NFκB inhibitor is the most effective dose on MV4-11 based on the FACS analyses. Three days after the one dose treatment, cells were then collected for either analyses by FACS and qPCR.

Transfection of siRNA-NFκB2 in MV4-11. A small interfering RNA (siRNA) was used to transiently knockdown NFκB2 (p100/52) in MV4-11 cells. Cells transfected with scramble siRNA were used as control (siRNA-NegControl). The cells were grown in 24-well plates and transfected with siTran 2.0 siRNA transfection reagent (Origene Cat #TT320002) using 50 nM NFκB2 (human, ID 4791) 27mer siRNA duplexes (#SR303162, Origene, Rockville, MD, USA). In experiments with GILT treatment, one dose of 80 nM GILT was added to MV4-11 cells with siRNA-NFκB2 in 24 well plates at the same time. The qPCR experiments including confirmation of knockdown and gene changes were performed 2 days after transfection.

Flow Cytometry (FACS). Cells were harvested and examined for the expression of cell surface biomarkers (CD) and intracellular proteins by multichromatic FACS as previously described [24]. About 1×10⁴˜10⁶ cells in 100 μl FACS buffer (PBS containing 1% FBS and 0.05% sodium azide) were stained with various fluorescence-conjugated antibodies specific for the desired cell surface proteins at 4° C. for 30 min. The surface-stained cells were then fixed and permeabilized using the appropriate reagents (e.g. the BD Pharmingen Cytofix/Cytoperm buffer) and stained with different fluorescence-conjugated antibodies specific for the desired intracellular proteins at 4° C. for 2 hours in the permeabilizing buffer (e.g. the BD Perm/Wash buffer). Concentrations of the Abs were used per the manufacturers' recommendations. Finally, the cells were washed twice in the permeabilizing buffer and twice in the FACS buffer before being analyzed on the BD FACSAria II. Data was analyzed using the FlowJo software (Tree Star Inc., Ashland, OR).

RNA Isolation and Real-Time Polymerase Chain Reaction (qPCR) analysis. MV4-11 and MOLM-14 cells were cultured with the presence of TKI drugs for 72 hours or re-plated 28 days after the treatment of TKI drugs. Cells were isolated for RNA isolation and qPCR analysis as previously described. Total RNA was isolated using the RNeasy Micro Kit (Qiagen) according to the manufacturer's instruction. First-strand cDNA was synthesized using the SuperScript III Reverse Transcriptase (Invitrogen; Life Technologies). With an Applied Biosystems 7900HT Real-Time PCR machine, qPCR was performed and analyzed using known primers for the specific markers. The PCR conditions were 10 minutes at 95° C. followed by 40 cycles of 10 seconds at 95° C. and 15 seconds at 60° C. The relative expression level of a gene was determined using the ΔΔCt method and normalized to GAPDH.

Proteomics and Blotting Analysis. Three days after the one dose treatment, the cell-free supernatants were collected for human cytokine profile assay. The Human XL Cytokine Array Kit (Catalog #ARY022B, R&D) was used according to the manufacturer's procedures and exposed to X-ray film (<10 minutes). Profiles of mean spot pixel density were created using a transmission-mode scanner and Image J analysis software.

Imaging Acquisition. Phase-bright images were taken using an Olympus 1X71 inverted microscope and were processed using an Olympus cellSens Dimension 1.15 Imaging Software.

Statistical Analysis. Statistical significance was assessed by ANOVA or by independent student “t” test for comparison between two groups. All values were presented as mean±SEM. Results were considered significant when the P value was <0.05.

### Subjects and Reagents

TABLE 3
List of Exemplary Reagents
List of Reagents

| | Abbreviation/Name | | | Species |
|---|---|---|---|---|
| Antibody/Reagents | in the text | Cat. # | Company | Reactivity |
| Viability Dye eFluor ™ 780 | Viability Dye | 65-0865-14 | eBioscience | |
| CD13-PE-CY7 | CD13 | 301712 | Biolegend | Human |
| CD33-PERCP | CD33 | 341640 | BD | Human |
| CD33-APC | CD33 | 303408 | Biolegend | Human |
| CD44-FITC | CD44 | 338804 | Biolegend | Human |
| CD44-PE/Cyanine7 | CD44 | 338816 | Biolegend | Human |
| CD117-PERCP | CD117 | 313214 | Biolegend | Human |
| Ki67-PE | Ki67 | 350504 | Biolegend | Human |
| Midostaurin | MIDO | M1323 | Sigma Aldrich | |
| Gilteritinib (ASP2215) | GILT | S7754 | SELLECK CHEM | |
| Quizartinib | QUIZ | A10027 | ADOOQ | |
| Sorafenib | SORA | SML2633 | Sigma Aldrich | |
| SB225002 | CXCR2-I | HY-16711 | MedChemExpress | |
| BAY11-7082 (BAY11-7821) | NFκB-I | S2913 | SELLECK CHEM | |
| Recombinant Human MIF | MIF | 300-69 | Peprotech | Human |
| RPS19 peptide | MIF-I | Orb217902 | Biorbyt | |
| Human XL Cytokine Array Kit | Cytokine Array | ARY002B | R&D SYSTEMS | Human |
| siRNA Oligo NFκB2 | siRNA-NFκB2 | SR303162 | Origene | Human |

TABLE 4
Characteristics of AML-FLT3 Patients

| No. | Diagnosis | Age | Sex | Disease Status | Gene Mutations |
|---|---|---|---|---|---|
| #1 | AML | 55 | M | Newly diagnosed | 1. FLT3: 40% allele frequency |
| | | | | August 2019 | (c.2503G > T; p.D835Y) |
| | | | | | 2. NPM1: SR 0.72 |
| | | | | | (c.863_864insCTTG; p.W288Cfs*12) |
| #2 | AML | 41 | M | Relapsed/Refractory | FLT3 Internal Tandem Duplication (ITD): |
| | | | | | Allele Frequency: |
| | | | | | (2 separate mutations) 0.25% & 0.52% |
| #3 | AML | 69 | M | Relapsed/Refractory, | 1. FLT 3 ITD: SR 0.89 |
| | | | | On treatment | (c.1789delins25; p.Y597delins9) (23% allele frequency) |
| | | | | | 2. RUNX1: 94% allele frequency |
| | | | | | (c.602G > A; p.R201Q) |
| | | | | | 3. IDH2: 65% allele frequency |
| | | | | | (c.419G > A; p.R140Q) |
| | | | | | 4. DNMT3A: 48% allele frequency |
| | | | | | (c.2645G > A; p.R882H) |
| | | | | | 5. SRSF2: 46% allele frequency |
| | | | | | (c.284_307del24; p.P95_R102del) |
| #4 | AML | 53 | F | Newly Diagnosed | 1. CBFB-MYH11: Allele Frequency 102.3453% |
| | | | | June 2019 | 2. FLT3 (c.1793_1794ins21; p.Y597_E598insDDPSLID): |
| | | | | | Allele Frequency 0.65% |
| | | | | | 3. KIT (c.1251_1257dekubsGGCA; p.Y418_D419delinsA): |
| | | | | | Allele Frequency 2% |
| #5 | AML | 76 | M | Newly Diagnosed | 1. DNMT3A (c.2645G > A; p.R882H): Allele Frequency 44% |
| | | | | February 2020 | 2. FLT3 (c.2503G > T; p.D835Y): Allele Frequency 41% |
| | | | | | 3. NPM1 (c.860_863dupTCTG; p.W288Cfs*12): NPM1 4 Base |
| | | | | | Pair Insertion confirmed with a Signal Ratio of 0.86 by PCR. |
| | | | | | 4. SF3B1 (c.1997A > G; p.K666R): Allele Frequency 46% |
| #6 | AML | 62 | M | Newly diagnosed | 1. FLT3-ITD: Level = 0.98 |
| #7 | AML | 65 | F | Refractory | 1. FLT 3: ITD SR 0.57 |
| | | | | | 2. PHF6: 41% allele frequency |
| | | | | | (c.821G > A; p.R274Q) |
| | | | | | 3. RUNX1: 34% allele frequency |
| | | | | | (c.494_497dup; p.G168Kfs*46) |
| | | | | | 4. WT1: 82% allele frequency |
| | | | | | (c.1140dup; p.S381Vfs*4) |

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A method of evaluating resistance to a tyrosine kinase inhibitor (TKI) therapy of acute myeloid leukemia (AML) in a subject having AML, the method comprising:
isolating AML tumor cells from the subject;
detecting expression of CD33, CD44, and phosphorylated BCL2 associated agonist of cell death (pBAD) on surface of the AML tumor cells; and
in response to the AML tumor cells being CD33 positive, CD44 high positive, and pBAD positive, the subject is determined to be at an increased risk of developing TKI-resistant AML.

2. The method of claim 1, wherein the CD33 positive, CD44 high positive, and pBAD positive tumor cells are Ki67 positive.

3. A method of treating acute myeloid leukemia (AML) in a subject, said method comprising:
administering a therapeutically effective amount of a TKI and a therapeutically effective amount of one or more inhibitors of a TKI-activated compensation pathway to the subject, thereby treating AML in the subject.

4. The method of claim 3, wherein the inhibitor inhibits a JAK-STAT pathway.

5. The method of claim 3, wherein the inhibitor inhibits a TYK2-STAT4-PIM2/PIM3 pathway.

6. The method of claim 3, wherein the inhibitor inhibits a NFκB2(P100/P52)-MIF-CXCR2 pathway.

7. The method of claim 3, wherein the one or more inhibitors are one or more of a STAT4 inhibitor, a BCL2 inhibitor, a CD44 inhibitor, a CXCR2 inhibitor, and an NFκB inhibitor.

8. The method of claim 3, comprising administering a therapeutically effective amount of each of a TKI, a BCL2 inhibitor, and a NFκB inhibitor to the subject.

9. The method of claim 3, comprising administering a therapeutically effective amount of each of a TKI, a STAT4 inhibitor, a BCL2 inhibitor, and a NFκB inhibitor to the subject.

10. The method of claim 3, wherein the TKI is Gilteritinib.

11. The method of claim 3, wherein the TKI is Quizartinib.

12. The method of claim 7, wherein the STAT4 inhibitor is (R)-Lisofylline.

13. The method of claim 7, wherein the BCL2 inhibitor is Venetoclax.

14. The method of claim 7, wherein the NFκB inhibitor is MG-132 or BAY 11-7082.

15. The method of claim 7, wherein the CD44 inhibitor is Angstrom6.

16. The method of claim 7, wherein the CXCR2 inhibitor is SB225002.

17. The method of claim 3, wherein the subject has TKI-resistant AML or is at risk of developing TKI-resistant AML.

18. The method of claim 3, wherein the subject has one or more mutations in FMS-like tyrosine kinase 3 (FLT3) gene.

19. A method of treating acute myeloid leukemia (AML) in a subject, said method comprising:
isolating AML tumor cells from the subject;
detecting expression levels of CD33, CD44, Ki67, and phosphorylated BCL2 associated agonist of cell death (pBAD) on surface of the AML tumor cells; and
in response to the tumor cells being CD33 positive, CD44 high positive, Ki67 positive, and pBAD positive, administering a therapeutically effective amount of a tyrosine kinase inhibitor (TKI) and a therapeutically effective amount of one or more inhibitors of a STAT4 inhibitor, a BCL2 inhibitor, a CD44 inhibitor, a CXCR2 inhibitor, and an NFκB inhibitor.

20. The method of claim 19, wherein the TKI is Gilteritinib or Quizartinib.
